(19) United States
(12) Patent Application Publication
(10) Pub. No.: US 2002/0010145 A1
(43) Pub. Date: Jan. 24, 2002

(54) Apparatus, methods and compositions for biotechnical separations

(76) Inventors: Richard C. Willson III, Houston, TX (US); Jason Murphy, Houston, TX (US)

Correspondence Address: RICHARD COALE WILLSON JR, 3205 HARVEST MOON DR, STE 200, PALM HARBOR, FL, 34683-2127, US

(21) Appl. No.: 09/841,763

(22) Filed: Apr. 24, 2001

Related U.S. Application Data
(63) Continuation-in-part of application No. 09/609,996, filed on Jul. 3, 2000.
(60) Provisional application No. 60/143,768, filed on Jul. 12, 1999.

(51) Int. Cl. A61K 48/00; C12N 15/63

(52) U.S. Cl. 514/44; 425/270; 435/320.1

(57) ABSTRACT

embodiments of the invention include purification of DNA, preferably plasmid DNA, by use of selective precipitation, preferably by addition of compaction agents Also included is a scaleable method for the liquid-phase separation of DNA from RNA. RNA may also be recovered by fractional precipitation according to the invention. RNA, commonly the major contaminant in DNA preparations, can be left in solution while valuable purified plasmid DNA is directly precipitated. Endotoxin can also be kept to very low levels. The invention includes mini-preps, preferably of plasmid and chromosomal DNA to obtain sequenceable and restriction digestible DNA in high yields in multiple simultaneous procedures. As a method of assay, a labeled probe is precipitated by hybridizing it to a target, (erg. chromosomal DNA, oligonucleotides, Ribosomal RNA, tRNA), and thereafter precipitating the probe/target complex with compaction agents and leaving in solution any unhybridized probe.

### BACKGROUND OF THE INVENTION

The present application is a continton-in-part of US Patent Application 091609,996 filed 0710312000, which itself has priority of US Provisional Applcation 60/143,768 filed 07/12/1999.

The RNA research was fimded in part by grants to R.C.W. and G.E.F. from the National Space Biomedical Research Institute, the Environmental Protection Agcy (825354-01-0), the Envirornentat Istie of Houston, the Robert A Welch Foundation, and the University of Houston/Shell Interdiscipnary Scholars Program.

I. Field of the Invention

The present invention relae to the gnrlfield of biochemical assays andseparations, and to appar forthei practice, generlly classified in U.S. Patent Class 435.

II. Description of the Prior Art

Interest in nucleic acid purification has increased with human trials of plasmid-based vaccmes (e gifor influenza, HIV, and malaria) and therapeutics (eg, insulin and vascularization on promoters) as well as the steady expansion of DNA sequencing activities (references 1 and 2) This invention embodies a rapid, scaeable s nucfre-fe (preferably RNAse free), cost effective method of nueic acid purification usig selective precipitation by compaction agents.

Prior Art will include the following:

1. Parasrampuria, D andHunt, ,(1998), Therapeutic issues in gene therapy; part 1: vectors. Biopharm. 11:38-45.

2. Anderson, F., (1998), Human Gene Therapy. Nature. 392: 25-30.

3. Horn, NA, M J.A, Budahazi, G., and Marquet M. 1995. Cancer gene therapy using plasmid DNA: purification of DNA for human clinical trials. Human Gene Therapy. 6:565-573.

4. Gosule, LC. and Schellman, J.A., (1976), Compact form of DNA induced by spermidine Nature. 259:333-335.

5. Arscott, P.G., Li, A.Z., and Bloomfield, V.A., (1990), Condensation of DNA by trivalent cations. 1. Effects of DNA length and topology on the size and shape of condeneed particles Biopolymers. 30:619-630.

6. Wilson, WW and Bloomfield, V.A., (1979), Counter-ion induced condensation of deoxyribonucleic acid. A light scattering study. Biochemistry. 18:2192-2196.

7. Bednar, J., Furrer, P., Stasiak A, Dubochet, J., Egelman, E.H., and Bates, A.D., (1994), The twist, writhe and overall shape of supercoiled DNA change during counterion-inde d transition from a loosely to a tightly interwound superhelix possible implications forDNA structure in vivo. Journal of Molecular Biology. 235:825-847.

8. Rolland, A., (1998), From genes to gene medicines: recent advances in nonviral gene delivery. Critical Review of Therapeutic Drug Carrier Systems. 15:143-198.

9. Hoopes, B.C. and McClure, W.R., (1981) Studies on the selectivity of DNA precipitation by spermine Nucleic Acids Research. 9:5493-5504.

10.Sambrook, J., Fritsch, E.f., and Maniatis, T., (1989), Molecular cloning, a laboratory manual. Second edition, Cold Spring Harbor Laboratory Press.

11.Horn, N., Marquet, M., Meek, J., and Budahazi, G., (1996), Process for reducing RNA concen on in amxture of biological material using diatomaceous eact U.S. Pat. No. 5,576,196.

12. Lev, Z., (1987), A procedure for la scale isolation of RNA-free plasmid and phage DNA without the use of RNAse. Analytical Biochemistry. 160:332-336.

13. Drevin, L,Larsson, L., and Johansson, B.L., (1989), Column performance of Q-Sepharose HP in analytical- and preparative-scale chromatography. Journal of Chromatography. 477:337-344.

14. Horn et al, U.S. Pat. 5,707812 Purification of Plasmid DNA During Column Chromatography, which is understood to teach addition of short chain polymeric alcohol to promote isolation of plasmid DNA.

15. Hubert, P, and Dellacherie, E, (1980), Use of water-soluble biospecific polymers for the purification of proteins, Journal of Chromatography, 184, 325-333.

16. Irwin, J.A., and Tipton, K.F., (1995), Affinity precipitation: a novel approach to protein purification, Essays in Biochemistry, 29, 137-156.

17. Widom, J., and Baldwin, R.L., (1983), Monomolecular condensation of λ-DNA induced by Cobalt Hexamemine, Biopolymers, 22, 1595-1620.

18. Nunn, C.S., and Neidle, S. 1996. The high resolution crystal structure of the DNA decamer d(AGGCATGCCT). J. Mol. Biol. 256:340-351.

19. Kieft, J.S. and Tinoco, I. 1997 Solution structure of a metal-hinding site in the major groove of RNA complexed with cobalt (III) hexammine. Structure. 5(5):713-721.

20. Pitulle, C, Hedenstierna K.O., and Fox G.E. 195 A novel approach for monitoring genetically engineered microorganisms by using artificial, stable RNAs. Applied Environment Microbiology. 61(10): 3661-3666.

21. Setterquist, R.A, SmithG.K., Oakley, T.H., Lee, Y.H., and Fox, G.E. 1996. Sequence, overproducton and purification of Vibrio proteolyticus ribosomal protein L18 for in vitro and in vivo studies. Gene. 183(1-2):237-242.

22. Yang, Y. and Fox, G.E. 1996. An Archa 5S rRNA analog is stablely expressed in Escherichia coli. Gene. 168: 81-85.

23. Sioud, M and Drlica, K. 1991 Prevention of human immunodeficiency virus type 1integrase expressionin Escherichia coli by a ribozyme. Proc. Natl. Acad. Sci. USA 88:7303-7307.

24. Couture, L.A. and Stinchcomb, D.T. 1996 Anti-gene therapy: the use of ribozymes to inhibit gene functon TIG. 12(12):510-514.

25. Christoffersen, R.E., and Marr J.J., (1995), Ribozmes as human therapeutic agents, Journal of Medicinal Chemistry, 38(12), 2023-2037.

26. Weiss, B., Davidkova, G. and Zhou L.W., (1999), Antisense RNA gene therapy for studying and modulating biological processes, Cell. Mol. Life Sci., 55, 334-358.

27. Kumar, M. and Carmichael G.G., (1998), Antisense RNA: function and fate of duplex RNA in cells of higher eukaryotes Microbiology and Molecular Biology Reviews, 62(4), 1415-1434.

28. Matthews, H.R., (1993), Polyamines, chromatin structure and transcription, BioEssays, 15(8), 561-566.

29. Hedemstierna, K.O.F., Lee, H.Y.,Yang, Y., and Fox, G.E, (1993), A prototye stable RNA identification cassette for monitoring plasmids of genetically engineered microorganisms. System. Appl. Microbiol. 16,280-286.

30. Pitulle, C, Dsouza, L., and Fox, G.E. 1997. A low molecular weight artificial RNA of unique size with multiple probe target regions. System. Appl. Microbiol. 20:133-136.

31. Uchiyama, S., Imamura, T., Nagai, S., and Konishi, K. 1981. Separation of low molecular weight RNA species by high-speed gel filtration. J. Biochem. 90:643-648.

32. Lee, K.M. and Marshall, A.G. 1986.High-speed preparative-scale separation and purification of ribosomal 5S and 5.8S RNA's via Sephacryl S-300 gel filtration chromatography Preparative Biochemistry 16(3):247-258.

33. Hori, S. and Ohtani, S. 1990. Separation of high-molecular mass RNAs by high-performance liquid chromatography on hydroxyapatite. Journal of Chromatography. 515:611-619.

34. Fair, W.R., and Wehner, N., (1971) Antibacterial action of spermine: effect on urinay tract pathogens, Applied Environmental Microbiology, 21(1), 6-8

35. Scopes, R.K., (1993) Protein purification: principles and practice, Springer-Verlag, 379 pages.

36.Blackburn, G.M., and Gait, M.J., (1996), Nucleic Acids in Chemistry and Biology, Oxford University Press, pages 337-346.

37. Saenger, W., (1988), Prnciples of Nucleic Acid Structure, Springer-Verlag, pages 432-434.

38. Ma, C., Sun, L., and Bloomfield, V.A., (1995) “Condensation of Plasmids Enhanced by Z-DNA Conformation of d(CG)ₙ Inserts”, Biochemistry, vol. 34(11), 3521-3528.

39. U.S. Pat. 5,622,822, to Tobias et al, Issued 1997 04 22, (Assigned Johnson & Johnson), Methods for capture and selective release of nucleic acids usig polyethylenemine and an anionic phosphate ester surfactant and amication of same teaches that nucleic acids can be made available for amplification or other treatment after lysis by contacting the lysate with polyethyleneimine to form a precipitate with the nucleic acids. The nucleic acids are then released from the precipitate by contact with a strong base, and the released nucleic acids are kept in solution with an anionic phosphate ester surfactant. Prior Art as to the RNA inventons includes [note some duplication for convenience.]:

40. Sambrook, J., Fritsch, E.F., and Maniatis, T. (1989) Molecular cloning, a Laboratory manual, Cold Spring Harbor Laboratory Press.

41. Hubert, P. and Dellacherie, E. (1980) Use of water-soluble biospecific polymers for the purification of proteins. J Chromatogr. 184, 325-333.

42. Irwin, J. A. and Tipton, K. F. (1995) Affinity precipitation: a novel approach to protein purification. Essays Biochem. 29, 137-156.

43. Hoopes, B. C. and McClure, W. R. (1981) Studies on the selectivity of DNA precipitation by spermine. Nucleic Acids Res. 9, 5493-5504.

44. Gosule, L. C. and Schellman, J. A. (1976) Compact form of DNA induced by spermidine. Nature. 259, 333-335.

45. Widom, J. and Baldwin, R. L. (1983) Monomolecular condensation of lambda-DNA induced by cobalt hexamie. Biopolymers. 22, 1595-1620.

46. Murphy, J. C., Wibbenmeyer, J. A., Fox, G. E., and Willson, R. C. (1999) Purification of plasmid DNA using selective precipitation by compaction agents.

Nature Biotechnol. 17, 822-823.

47. Nunn, C. M. and Neidle, S. (1996 The high resolution crystal structure of the DNA decamer d(AGGCATGCCT).) J. Mol. Biol. 256, 340-351.

48. Setterquist, R. A., Smith; G. K., Oakley, T. H., Lee, Y. H., and Fox, G. E. (1996) Sequence, overproduction and purification of Vibrio proteolyticus ribosomal protein L18 for in vitro and in vivo studies. Gene. 183, 237-242.

49. Pitulle, C., Hedenstierna, K. O., and Fox, G. E. (1995) A novel approach for monitoring genetically engineered microorgasms by using artificial, stable RNAs. Appl.Environ.Microbiol. 61, 3661-3666.

50. Pitulle, C., DSouza, L., and Fox, G. E. (1997) A low molecular weight artificial RNA of unique size with multiple probe target regions. Sys. Appl. Microbiol. 20, 133-136.

51. Sioud, M. and Drlica, K. (1991) Prevention of human immunodeficiency virus type 1 integrase expression in Escherichia coli by a ribozyme. Proc. Natl. Acad. Sci. U.S.A. 88, 7303-7307.

52. Fair, W. R. and Wehner, N. (1971) Antibacterial action of spermine: effect on urinary tract pathogens. Appl. Microbiol. 21, 6-8.

53. Arscott, P. G., Li, A. Z., and Bloomfield, V. A. (1990) Condensation of DNA by trivalent cations. 1. Effects of DNA length and topology on the size and shape of condensed particles. Biopolymers. 30, 619-630.

54. Wilson, R. W. and Bloomfield, V. A. (1979) Counterion-induced condensation of deoxyribonucleic acid. a light- scattering study. Biochemistry. 18, 2192-2196.

55. Kieft, J. S. and Tinoco, L, Jr. (1997) Solution structure of a metal-binding site in the major groove of RNA complexed with cobalt (III) hexammine. Structure. 5, 713-721.

II. Problems Presented by Prior Art

Most current methods of plasmid separation are relately time-consuming and require the use of adsorbents, toxic substances, nucleases, and/or filtration media to separate plasmid from protein genomic DNA, endotoxims and especally the abunant RNA present im cell lysates.

This technique offers several important improvements over current methods: no RNAse and/or other enzymes are used, the technique requires no chromatographic medium, and the technique is directly scaleable if larger quantities of plasmid DNA are needed.

Also with the use of different compaction agents, different types of nucleic acids canbe separated the same mixture. The invention can separate different types of RNA and DNA as long as some secondary structure is present.

In addition, RNA can be fractionated based on molecular weight via selective precipitation.

Different compaction agents also have different affinities for different nucleic acids. For example hexammine cobalt has a higher affinity for RNA than the polyamine spermidine so multiple step selective precipitations have been developed to help separate nucleic acids as quickly as possible.

The method can also be used for parallel purification of a large number of samples (mini-preps) and is readily adaptable to automation (robotics).

In another embodiment, the invention alo provides a method for making a biochemical assay by hybridizing a labeled probe to a target (e.g. chromosomal DNA, olgonucleotides, ribosomal RNA, tRNA, plasmid, aptamer, viral RNA), an thereafter precipitating the probe/target complex with compaction agents. For example, preparing a mixture containing chromosomal DNA, plasmid, ribosomal RNA, and labeled oligonucleotides, then heating the mixture of nucleic acids above their melting temperature (if the hybridizaton site is buried within secondary structure) and thereafter precipitating the probe and the target),

In another embodiment, the invention also provides a method for separating a nucleic acid-binding protein from a mixure containing the protein and its nucleic acid binding partner and of components, by precipitating the bound nucleic acid carrying the associated protein into the precipitate, from which it may optionally be further purified. For example a selected protein might be isolated from culted human cells containing both the protein and a DNA sequence to which the protein binds, by making a lysate from the cells and precipitating the DNA, producing a precipitate enriched in both the DNA target sequence and in the binding protein.

Bioseparations, especially separation of RNA from DNA or vice versa, are conventionally accomplished in bench scale or pilot plants in which a fermentation is carried out to produce cell mass which is lysed, then exposed to filtration and nucleases are used to reduce unwanted nucleic acid populations (e.g. the use of ribonuclease (RNAse) in plasmid purification). Generally, after these initial solution phase purification steps, the effluent products are purified by chromatographic columns (e.g. anion-exchange or size-exclson chromatography), often with samples being analyzed and result subjected to quality control feedback techniques. Such procedures can take a day or more for a single run or batch on a single mixture, assuming the optimum conditions, concentrations, etc. The present invention permits the separation of dozens of feed mixtures in a single set-up, often in less time than required for a single separation by conventional methods. Further, when practiced in its preferred embodiments, the invention can sharply reduce the production costs (costs per miligram of purified DNA product produced).

In addition, the labeled probe precipitation embodiment offers a new method for hybridization assays without the use of radiolabeled probes or the use of solid supports. Using compaction precipitation, when a tagged probe (e.g. fluoresceinated, radioactively tagged, etc.) is added to a solution containing its target a double stranded nucleic acid is formed and this new structured hybrid can be selectely precipitated while the single-stranded probe will be left in solution.

In addition, the nucleic acid/binding protein coprecipitation embodiment offers a new method of identifying and/or separating nucleic acid-binding proteins from cells expressing them. Using compaction precipitation, these proteins can be selectively precipitated away from other proteins, producing a significant degree of selective enrichment without the need to prepare costly affinity adsorbent matrices.

Summarizing, preferred embodiments include the assay, the protein purification, and selectivity for DNA precipitation over RNA, isolation of RNA by first precipitating DNA, then separately precipitating RNA in a second step, and isolation of RNA by first precipitating DNA, then separately fractionating (precipitating) large RNA molecules in a second step, finally precipitating low molecular weight RNA with a third precipitation step.

### SUMMARY OF THE INVENTION

General Statement of the Invention

According to the invention, in preferred embodiments, DNA, preferably plasmid DNA is readily purified, by use of selective precipitation, preferably by addition of compaction agents. Also, included is a scaleable method for the liquid-phase separation of DNA from RNA. RNA may also be recovered by fractional precipitation according to the invention.

We have discovered RNA, commonly the major contaminant in DNA preparations, can be left in solution while valuable purified plasmid DNA is directly precipitated.

Additional aspects of the invention included mini-preps, preferably of plasmid and chromosomal DNA to obtain sequenceable and restriction digestible DNA in high yields in multiple simultaneous procedures.

Still further aspects disclose enhanced stripping of the compaction agent by a stripping method comprising high salt addition or pH shift, and combinations of these techniques.

Also, disclosed is a method of assay in which a labeled probe is precipitated when it is hybridized to a target, (e.g. chromosomal DNA, oligonucleotides, ribosomal RNA, tRNA and thereafter precipitating the probe/target complex with compaction agents and leaving in solution any unhybridized probe. For example, chromosomal DNA, plasmid, ribosomal RNA, and oligonucleotides can be recovered in excellent purity; by then heating the mixture of nucleic acids and probe (above their melting temperature if the hybridization site is buried within secondary structure) and thereafter precipitating the probe and the target whereby the target can be detected.

Further disclosed is a method for producing a reduced-viscosity cell lysate, useful as a starting point for further purification of product by removal of nucleic acids through compaction precipitation.

Each of these parameters is discussed below:

A new method for DNA separation has been developed using selective precipitation with small-molecule compaction agents, such as spermine and spermidine, which bind in the grooves of a double-stranded DNA molecule. Compaction precipitation uses compaction agents to neutralize the highly charged phosphate backbone of nucleic acids and to stabilize intermolecular interactions leading to precipitation. This selective precipitation has been demonstrated to separate double-stranded plasmid DNA from RNA, protein and other contaminants in solution. Using compaction precipitation, we have also developed an improved mini-prep procedure capable of producing sequencing-grade plasmid DNA. The precipitation of nucleic acids from lysates can also be applied to the clarification of protein lysates before any subsequent chromatography is done.

In addition, a compaction agent-based selective precipitation of RNA from clarified lysates of bacteria, fungi, or metazoan cells and/or mixtures of biomolecules has been developed, The use of selective precipitation with compaction agents and anion-exchange chromatography have been shown to effectively separate the ribosomal RNA's from each other and 5S rRNA from tRNA. Compaction agent-based separation of RNA produces either a total RNA mixture or a high molecular weight RNA fraction with little contaminating protein or DNA. Anion-exchange chromatography can then be used to separate the different RNA molecules from the total bacterial RNA sample. Also, using compaction precipitation and labeled oligonucleotide probes, a hybridization assay has been developed for use in a wide variety of applications, including e.g. environmental monitoring. quality control of nucleic acids, medical diagnostics, and use in mutation studies.

Still another embodiment comprises isolating nucleic acid-binding proteins by coprecipitating them with the nucleic acids to which they bind. This method can be used in purification and identification of regulatory proteins, histones, and aptamers, for example.

Cell Mass: The starting material is often a mass of cells prepared by fermentation or cell culture, isolated from the environment, or derived from tissues. The cells are then disrupted so the nucleic acids go into solution, forming a lysate. The lysate then optionalty undergoes an alkaline lysis or other process to form a clarified lysate. The preferred feed to the compaction precipitation step is a clarified lysate or synthetic mixure. A variety of cell types can be used as feed for this whole process, with bacterial, yeast, other eukryotic, Gram-negative and Gram-positive being preferred and Gram-negative being most preferred.

Product: The product of the invention can be purified DNA, RNA or nucleic acid-binding proteins, preferably DNA, and most preferably plasmid DNA, e.g. as used in preparation of influenza or other vaccines. Alternative preferred product is RNA, preferably ribosomal RNA, ribozymes, aptamers, artificial RNA, and any other RNA based molecule.

Particularly preferred is RNAse-free plasmid having a quantity of nucleases below current limits of detection and/or low endotoxin contamination. In other embodiments, the product can be a bioassay or protein, e.g. as produced in Examples 13 and 16.

In general, the selective precipitation of the invention can be applied to all bacteria (Gram-negative, Gram-positive and Archaea), all eukaryotes (such as yeast and human cells), recombinant cells, and all synthetic nucleic acids. The invention can separate YAC's (yeast artficial chromosomes). YACs are very large plasmids in yeast, used in sequencing projects. The invention can also be applied to the production of cosmids and bacterial artificial chromosomes(basically very large plasmids in general), artificial chromosomes, a phage and other viral DNA, and the detection of protein-nucleic acid binding and viruses.

Compaction Agents: The compaction agents are preferably small, cationic molecules, which bind in either the major or minor grooves of a double-stranded RNA or DNA molecule, reducing the volume occupied by the nucleic acid FIG. 1 shows the structures of some common compaction agents).

Some compaction agents function in vivo to package genomic DNA into sperm (see reference 7), and can also serve a similar function in the delivery of DNA pharmaceuticals. (See reference 8).

Compaction of DNA involves charge neutralizatoin in combination with stabilization of inter-helix interactions. The compaction agent binds in either the major or minor groove, in proximity to the negatively charged phosphate groups. Precipitation occurs when adjacent DNA helices are affected simultaneously, with the compaction agent not only reducing the helix-helix repulsion but also bridging the helixes. Hoopes described this phenomenon in 1981 (see reference 9) but upon further investigation, we have discovered that RNA is far less readily precipitated by certain compaction agents, preferably linear polyamine type compaction agents, and found that RNA can be selectively precipitated and even fractionated using specialized compaction agents, most preferably, hexammine cobalt as the compaction agent and/or without substantial precipitation of contaminating endotoxins.

In general, there will be added about 0.1 to 20, more preferably about 0.2 to 15 and most preferably about 0.3 to 5 mM of a compaction agent, preferably selected from the group consisting of basic polypeptides (e.g. polylysine), polyamines (e.g. protamine, spermidine, spermine, putrescine, cadaverine, etc.), trivalent and tetravalent metal ions (e.g. hexammine cobalt, chloropentammine cobalt, chromium (III)), netropsin, distamycin, lexitropans, DAPI (4′,6 diamino 2-phenylindol), berenil, pentamidine, manganese chloride.) At present knowledge, the moieties in parenthesis will be more preferred, but any other molecule that can be used to compact DNA via the mechanism described above can be used according to the product to be produced and the cell mass available.

Many other agents may be considered compaction agents and these include: basic polypeptides (i.e. polylysine), polyamines (i.e. protamine, spermidine, spermine, cadaverine, etc.), trivalent and tetravalent metal ions (i.e. hexammine cobalt, chloropentammine cobalt, chromium (III)), netropsin, distamycin, lexitropans, DAPI (4′, 6 diamino 2-phenylindol), berenil, pentamidine, manganese chloride, or any other molecule that can be used to compact DNA via the mechanism described above (see references 1-7, 9,17-19, 36,37,38) Also any protein having multiple binding domains for nucleic acids can potentially, for large complexes, result in the precipitation of nucleic acids.

For the separation of plasmid DNA, genomic DNA, and other large double-stranded nucleic acids, the most preferred compaction agent is spermidine. It has a relatively low affinity for RNA (as determined my light scattering monitored condensation curves shown in Figures 11- 13.) yet has a high affinity for plasmid and other linear double-stranded DNA molecules.

For the separation of RNA the most preferred compaction agent is hexammine cobalt. It has a relatively high RNA affinity yet it behaves in a manner where it can be removed (stripped) from the RNA molecules without degradation to the RNA and relatively quickly.

For a total nucleic acid precipitation spermidine is the most preferred compaction agent because it has a relatively similar affinity for RNA, plasmid DNA and other nucleic acid molecules with some secondary structure. This is useful for example, when removing nucleic acids from a protein lysate.

Preferred Compaction Agent Selectivities

Light scattering-monitored condensation curves for plasmid DNA, salmon sperm DNA and total Vibrio proteoyticus RNA are shown in Figures 11-13. Spermidine has high potency for the condensation of plasmid DNA and chromosomal DNA but not RNA, hexxammine cobalt has a relatively broad scattering curve for total RNA, suggesting the possibility of fractionation, and spermine has a high potency for all three nucleic acids.

These scattering curves were used as the basis of a multi-step selective precipitation protocol for RNA in which plasmid DNA and chromosomal DNA are removed with an imitial spermidine precipitation, RNA is precipitated or fractionated with a hexammine cobalt precipitation, and small RNAs (<500 bases) can be precipitated at increased hexammine cobalt concentration.

To quantify more subtle differences in precipitation potency, we define a plasmid DNA/RNA selectivity ratio as the charge equivalents of compaction agents needed to condense plasmid DNA (to 95% of maximum observed signal) divided by the charge equivalents of compaction agent needed to condense total RNA to the same degree. Hexammine cobalt has a selectivity ratio of 0.34, which is lower that of spermine (0.83) and both, however, are significantly higher than that for spermine (taken to be zero as spermidine does not precipitate RNA up to 700 charge equivalents). The gradually rising condensation curve of hexammine cobalt (figure ***13) indicates the ability to fractionate total RNA by changing hexammine cobalt concentration so it was used even though spermidine has a high affinity for RNA. In addition, since hexammine cobalt has a +3 charge mstead of the +4 charge of spermine, hexammine cobalt is easier to remove from the necleic acids after precipitation has occurred.

Condensation Experiments:

Condensation curves were used to determine selectivities of compaction agents for different nucleic acids. A SPEX Fluorolog-2 Fluorometer was used with L-format excitation and emission wavelengths set to 500 nm. To 3 mL of 10 μg/ml nucleic acid, compaction agents were added with constant stirring in a series of aliquots at 210-second intervals until scattering intensity was constant.

Compaction Agent Selectivities:

The action of compaction agents on nucleic acids has previously been characterized using light scattering, FTIR difference spectroscopy, and NMR (Arscott et al., 1990; Wilson and Bloomfield, 1979) Hexammine cobalt is used extensively in NMR studies because of its high number of identical protons. It can be used to induce a B to Z transition in nucleic acids (Reich et al., 1994), (Kieft and Tinoco, Jr., 1997).

Light scattering-monitored condensation curves for plasmid DNA, salmon sperm DNA and total Vibrio proteoyicus RNA (purified by a 2 mM hexammine cobalt precipitation) are shown in FIGS. 11-13. Spermidine has high potency for the condensation of plasmid DNA and chromosomal DNA but not RNA. Hexammine cobalt has a relatively broad scattering curve for condensation of RNA. Finally, spermine has a high potency for precipitation of all three nucleic acids These scattering curves are used to design a multi-step selective preciptaion protocol for RNA in which plasmid DNA and chromosomal DNA are removed with an initial spermidine precipitation, RNA is precipitated or fractionated with a hexammine cobalt precipitation, and small RNAs (<500 bases) can be precipitated at increased hexammine cobalt concentration.

To quantify more subtle differences in precipitation potency, we define a plasmid DNA/RNA selectivity ratio as the charge equivalents of compaction agents needed to condense plasmid DNA (to 95% of maximum observed signal) divided by the charge equivalents of compaction agent needed to condense total RNA to the same degree. Hexammine cobalt has a selectivity ratio of 0.34, which is lower than that of spermine (0.83) and both, however, are significantly higher than that for spermidine (taken to be zero as spermidine does not precipitate RNA up to 700 charge equivalents). The gradually rising condensation curv of heammine cobalt (FIG. 13,) indicates the feasibility of fractionation of total RNA by changing hexammine cobalt concentration so it was used even though spermidine has a high affinity for RNA. In addition, since hexammine cobalt has a +3 charge instead of the +4 charge of spermine, hexammine cobalt is easier to remove from the nucleic acids after precipitation has occurred.

These selectivity experiments show why spermidine is, for many cases, a more useful DNA affinity precipitant then other commercially available compaction agents. Spermidine will not precipitate structured RNA (at least up to the level of 700 charge equivalents) because of the spread out +3 charge of the cation which leads to its relative impotency with RNA, and thus we have found that spermidine can be used to purify DNA without further digestion with nucleases (specifically RNAse).

Other Reagents:

Fluorescein Dyes: These include all fluorometric and colorimetric dyes. Examples of fluorometric dyes are Texas Red, and others well-known to the literature. In the assay application, we prefer probes labeled with fluorescein and other fluorescent dyes, or with enzymes which can be sensitively detected by adding chromogenic, fluorogenic, or chemiluminescent substrates. Fluorescent dyes are especially preferred, such as fluorescein. Enzymes compatible with chemiluminescent detection are also especially preferred, such as peroxidase and alkaline phosphatase.

Lysis solution: Examples include: alkaline lysis solutions, lysozyme containing solution, etc.

Resuspension solutiom A low ionic strength solution for resuspension of a nucleic acid precipitate before performing compaction precipitation. For example, 10 mM Tris HCl at ph 8.0. Compaction agent solution: A solution containing the appropriate concentration of a compaction agent to perform a precipitation (selective or non-selective based on application).

Stripping solution: A solution or combination of solutions used to remove compaction agents from compaction precipitated DNA. The most preferred solution for this contains 50% EtOH, 300 mM NaCl, and 10 mM EDTA. The important concepts here are the alcohol that causes the plasmid to stay precipitated (PEG 8000 can also be used here). Also the sodium chloride is used to procide a high ionic strength solution to remove the spermidine from the backbone. Alternatives to NaCl are KCI MgCl₂, or any other salt that raises ionic strength. EDTA is used as a chelating agent that binds free metals and compaction agents in solution. Alternates include EGTA, etc. EDTA (ETHYLENEDIAMINETETRAACETIC ACID): other possible chelating agents include:

Nitrilotriacetic acid, NTA: N(CH2COOH)3

Hydroxyethylethylenediaminetriacetic acid, HEDTA:=20

(HOOCH2C)2NCI2CH2N(CH2COOH)(CH2CH2OH)

Diethylenetriaminepentaacetic acid, DTPA:=20

(HOOCH2C)2NCH2CH2N(NCH2COOH)CH2CH2N(CH2COOH)2

1,2-Diaminopropanetetraacetic acid, 1,2-PDTA

(HOOCH2C)2NCH(CH3)CH2N(CH2COOH)2

1,3-Diaminopropanetetraacetic acid, 1,3-PDTA:

(HOOCH2C)2NCH2CH2CH2N(CH2COOH)2

2,2=B4-Ethylenedioxybis[ethyliminodi(acetic acid)], EGTA:=20

(HOOCH2C)2NCH2CH2OCH2CH2OCH2CH2N(CH2COOH)2

Bis(carboxmethyl)diaza-18-crown-6,

(HOOCH2C)N(CH2CH2OCH2CH2OCH2CH2)2N(CH2COOH)

1,10-bis(2-pyridylmetyl)-1,4,7,10-tetraazadecane, BPTETA:=20

(C6H4N)CH2NHCH2CH2NHCH2CH2NHCH2CH2NHCH2(C6H4N) and all other similar chelating agents

Also, combinations of the above components, for example omitting the EDTA or other chelating agent from the stripping solution. In addition, the solution can be broken into components and then added step-wise as multiple solutions. For example, a high ionic strength solution possibly with a chelating agent can be added to the pellet then an alcohol solution or PEG containing solution could be used to then precipitate and desalt the solution by precipitating the nucleic acids so the salt containing supernatant can be poured off.

Final resuspension solution: This is preferably 10 mM Tris HCl with 1 mM EDTA at pH 8.0 (TE). It can be any solution in which the user desires to resuspend the purified nucleic acid.

RNA lysis solutions: These include nonionic detergents Brij 58, Brij 99, etc. and alo commercial mixtures of nonionic detergents such as BPER from Pierce and Bugbuster from Novagen. The lysis solution can be used separately from or combine with a compaction agent solution to precipitate unwanted DNA. The second compaction agent solution is at an appropriate concention to precipite RNA. The stripping solution can be the same as in Example 26, except that 6 M urea (or an equivalent denaturation solution) can preferably be included.

Lysing Agents: Lysing agents, preferably detergents, more preferably nonionic detergents, are used to break down cell membranes, thus releasing DNA, RNA, and proteins from the cells. The most preferred lysing agent for plasmid DNA is the akaline lysis detailed in Example 1. The most preferred lysing agent for RNA is Bacterial Protein Extraction Reagent (BPER) which has an unknown composition (it is a proprietary mixture of nonionic detergents marketed by the Pierce Chemical Company), but other nonionic detergents are useful and many detergents are operable, even some anionic and cationic detergents under certain applications. We have found that the nonionic detergent brij 58 is a useful alternative to BPER. The nonionic detergent lysing agents wil generally be added to the cell mass in a concentration of about 0.1 to 5, more preferably 0.5 to 2 wt. %. Other known lysing agents can also be used with the technology such as freeze/thawing, French cell press, enzymes, microfluidization, sonication, etc.

Nucleases: One of the main advantages of the compaction precipitation technology is that it circumvents the need to use nucleases, proteases or carbohydrases. Selective precipitation directly harvests nucleic acids and the target nucleic acid of a precipitation can be changed by changing conditions (i.e. type of compaction agent, quantity of compaction agent, concentration of salts, etc.) Because of this selectivity other large biomolecular contaminants such as proteins, unwanted nucleic acids, carbohydrates, etc. do not have to be degraded by enzymes. Thus the use of RNAse, DNAse, proteases, and other enzymes is unnecessary.

pH:All Examples are carried out at a pH between 6-8, to keep nucleic acid degradation to a minimum, though other pHs may be preferred in certain cases. The compaction agents can be affected by extreme pH. In fact, we have found that pH change (e.g., shifting the pH past the pK_{A} of the amine groups in polyamines, so that they lose their positive charge and do not bind nucleic acids strongly is one of the ways to separate nucleic acids from the compaction agents themselves.)

Ionic Strength: High ionic strength can negate the effects of compaction agents The preferred maximum ionic strength for compaction precipitation is 250 mM NaCI when plasmid is precipitated in 10 mM spermine. More preferred ionic strength before compaction agent addition is about 0-50 mM, more preferably 1 to 20 mM but those skilled in the art will adjust the ionic strength to best suit the particular lysate and compaction agents being employed. Changing ionic strength is an easy way to separate the compaction agents from the nucleic acids, because in the presence of a high ionic strength solution the compaction agents are displaced from the nucleic acid backbone.

Hybridizing: To hybridize means to bind to its complementary sequence in the target. If the probe used in a bioassay includes a sequence 5′-AAGC-3′; its hybridizing complementary sequence will be 5′-GCTT-3′. This is important because this test can be run as a valuable quality control measure on oligonucleotides and other synthetic nucleic acids, or used for detection of particular nucleic acid sequences and/or viruses in cells or tissues.

Batch or Continuous Conditions: The invention can be performed in commercially available equipment under batch or, less preferably, continuous flow stream, conditions, at elevated, reduced or atmospheric pressure and temperature, but atmospheric pressure and near ambient temperatures will be preferred for most applications.

Most large-scale bioseparation are done in batch because of the need to grow cells and the difficulty of maintaining a steady flow of cells from a chemostat, also the preparation wil preferably be conducted under 50 degrees C and more preferably under 25° C.

Description of Exemplary Kits,

The kits for practice of the methods of the invention preferably have somewhat different forms depending on their intended functions.

Plasmid DNA mini-prep kit:

These kt will preferably include a set of three common alkaline lysis buffers as described in the Qiagen product manual and in Sambrook as Solutions I, II, and III (25 mM Tris HCI with 10 mM EDTA at pH 8.0, 1% SDS and 0.2 N NaOH, and 3 M potassium acetate at pH 5.5 respectively), a resuspension solution (10 mM Tris HCl at pH 8.0), a compaction agent-containing solution (<2.9 mM spermidine trihydrochloride (from Sigma Chemical Co., product number 233994), a stripping solution (300 mM NaCI with 10 mM EDTA in 50% ethanol), and a final resuspension soluion (preferably TE which is 10 mM Tris with 1mM EDTA at pH 8.0. The resuspension and compaction agent solutions may be combined so that the IPA pellet from the lysis solution can be directly resuspended in a compaction agent containing solution such that the RNA and other contammants are extracted from the pellet without fully resuspending the IPA pellet. Also, centrifuge tubes or microfuge-based spin filters may also be included.

The kits will be packaged in plastic bottles and solution volumes will vary based on the amount of mini-preps for which the kit is rated. Also, centrifuge based spin filters can also be used in the separation. These can take advantage of the precipitates forming particles large than the pore size of commonly available microfuge base centrifuge spin filters. One model spin filter that works for this application is a Millipore Durapore centrifuge filter with a 0.45 mm pore size (Millipore corporation, catalog number ufc3 0hv 25. In addition, filters can be used that have a packed steel wool, cellulose or polymer/plastic material in a centrifuge or larger filer. Packed filters would not only be cost effective but may also work more efficiently for this application (they will not plug as easily).

Also, the lysis can be performed by a single solution lysis using lysozyme, a non-ionic detergent or other lysis means. Then the kit would comprise one bottle of lysis solution, one bottle of compaction agent solution (e.g. 29 mM spermidine in 10 mM Tris at pH 8.0), one bottle of stripping solution (e.g 50% EtOH with 300 mM NaCl, and 12.5 mM EDTA), a wash solution (namely 70% EtOH could be used or called for) and a final buffer for resuspension (usually TE at pL 8.0) Also, depending on the kit, other materials such as spin filters or centrifuge tubes could be included in this kit.

Large-scale Plasmid Prep kit:

This kit will include the same solutions as above but in larger quantity. Also, a vacuum based filtration setup can be used instead of centrifuge-based columns. (This is also a possibility with the small scale kits if a vacuum manifold or other vacuum system is employed).

RNA mini-prep kit:

The RNA mini-prep kit will typically consist of a solution of (preferably) nonionic detergent (eg bactenial proteine extraction reagent (e.g. from Pierce Chemical at a 2X dilution), a 1% solution of the non ioic detergent Brij 58, or any other lysis solution that will work in this system) with a novel amount of compaction agent(e.g 2.5 mM spermidine buffered in 10 mM bis tris propane at pH 6.9). This solution can be used to lyse bacteria (or other type of cell) and precipitate any DNA (using the spermidine) in one step.) Then a solution of hexammie cobalt (or possibly another compaction agent) will be used in a second RNA specific precipitation. A stripping solution, a wash buffer, and a final resuspension solution may also be included. Also, as with the plasmid DNA mini-prep, centrifuge based spin filters can also be used in the separation. These can take advantage of the precipitates forming particles large than the pore size of commonly available microfuge base centrifuge spin filters. If the initial lysate is filtered 2 microcentrifuge filters can be included per prep. If a second hexamine cobalt precipitation is done to capture small RNA fragments, an additional microfuge spin filter column may be included.

Filter Media:

A preferred material for these spin filter column filters is 0.45 μm pore size cellulose acetate membrane (e.g. Corning Filter System (we use 200 mL units but a wide variety of sizes are available) 0.45 μm cellulose acetate, model number 25933-200) since the material has an negligible affinity for biomolecules (specifically nucleic acids) and since it is a readily-available filter material. Ceramic filters can also be used. Also, a filter aid, such as a diatomaceous earth or similar compound, may also be used to the same end. For larger scale applications, a tangential-flowfilter can be used.

Chromosomal DNA kit

Another possible kit based on compaction precipitation is for the separation of genomic DNA from both eukaryotes and prokaryotes. The preferred lysis method is using lysozyme, protease K, with some EDTA and nonionic detergents to aid in the destruction of the cell membrane. In addition, other lysis techniques may be usefull with thits technique if undamaged genomic DNA is released during the course of the procedure. Next, an IPA precipitation can be done to desalt the solution and the a compaction precipitation using a resuspension solution (10 mM Tris HCl at pH 8.0), a compaction agent-containing solution (≦2.9 mM spermidine trihydrochloride (from SigmaChemical Co., product number 233994), a stripping solution (300 mM NaCl with 10 mM EDTA in 50% ethanol), and a final resuspension solution (preferably TE which is 10 mM Tris with 1 mM EDTA at pH 8.0).

II. Utility of the Invention

The present invention is useful in the separation of DNA from RNA and vice versa With numerous gene therapy products entering clinical trials, new and innovative strategies are needed to produce pure plasmid DNA.

In addition, with the advances in gene chips in which DNA is attached to a small piece of glass (so that one chip can have over 1 million nucleic acid probes and can be used to test for disease) and genetic diagnostics, environmental monitoring, ribozyme research, and aptamers, improved separation processes for nucleic acid molecules are in demand.

The separation of RNA from bacterial cells is conventionally achieved by phenol/chloroform extraction and polyacrylamide gel electrophoresis.

However, this conventional use of organic solvents and polyacrylamide (a neurotoxin) creates hazardous waste, and this approach is not easily scaleable for medium to large-scale production of RNA.

Selective precipitation by use of compaction agents acording to the present invention provides lower cost, more effective, and faster separation than the conventional methods of plasmid production (See refeences 10 and 14) An added unexpected advane of the selective precipitation of the invention is that it also contributes to improved performance of subsequent chromatographic columns used for further separation and purification. Of considerable value in production of pharmaceuticals, the invention permits the precipitation of plasmid DNA containing less than 0.1 Units endotoxin per microgram plsmid DNA (EU/μg or IE/μg). The kits described are exemplary of kits which can substantially ease and speed the separations and tests of the invention.

Additionally, various types of DNA and RNA can be separated. Using 3.5 mM hexammine cobalt, total RNA can be selectively precipitated from a cell lysate and at a concentration of 2 mM hexammine cobalt, rRNA can be fractionated from low molecular weight tRNA and mRNA. The resulting RNA mixture was readily resolved to pure 5S and mixed 16S/23S rRNA by nondaturing anon-exchange chromatography. Using a second stage of precipitation at 7.1 mM hexammine cobalt, the low molecular RNA weight fraction can be isolated by precipitation. Compaction precipitation is also applied to the purification of an artificial stable RNA derived from Eschefichia coli 5S rRNA and to isolation of an Escherichia coli expressed ribozyme.

### BRIEF DESCRIPTION OF THE DRAWINGS

FIG. 1 is a schematic diagram of preferred structures of common compaction agents

FIG. 2 shows schematically the precipitation by spermidine of 40 μg/mL pBGS19uxwt or Baker's yeast RNA in 10 mM Tris buffer at pH 8.0 with and without 600 mM NaCL. (Error bars are +/− one standard deviation.)

FIG. 3. Depicts a 1% agarose gel tracing the large-scale purification of pBGS19luxwt plasmid DNA. Lane 1 is a supercoiled plasmid ladder from Gibco; Lane 2 is the preparation after Celite filtration, isopropanol precipitation, and resuspension; Lane 3 is the supernatant after LiCl precipitation; Lane 4 is the supernatant the compaction precipitation by 2.9 mM spermidine HCl; Lane 5 is the resuspended pellet of the compaction precipitation after stripping of spermidine by 300 mM NaCl, 10 mM MgC12, and 25 mM EDTA in 50% isopropanol; Lane 6 is a 10X loading of the material in Lane 5 (The traces of genomic DNA in these lanes can be removed by further optimization of the initial lysis and preciptation steps); Lane 7 is after a Q Sepharose anion-exchange column (See FIG. 4, bottom, Peak 5); Lane 8 is a 10X loading of Lane 7 and Lane 9 is the same as Lane 1.

FIG. 4. Shows the chromatograms from a Pharmacia FPLC System using a HP Q Sepharose anion-exchange separaton of pBGS19luxwt of an alkine lysate after isopropanol and LiCl precipitation and optional compaction precipitation Top: NaCl gradient; Middle: with no previous compaction precipitation steps; Bottom: identical separation after a compaction precipitation step (1 volume of 2.9 mM spermidine in 10 mM Tris HCl at pH 8.0; see example 1). A Spectrum chromatography column (2.5 cm×60 cm) packed with 150 mL Q Sepharose high performance media and equilibrated in 10 column volumes of TE with 570 mM NaCl is used. Loading and elution are performed at a linear velocity of 90 cm/hr.

FIG. 5 shows schematically the process steps for separation of DNA as disclosed in Example 1.

FIG. 6 shows a 3% Biogel (from Bio101 Inc.) electrophoretic analysis of V. proteolyticus RNA purified by Example 9. Lane 1 is the Ambion RNA Century Plus Size Markers; Lane 2 is the lysate after BPER addition, spermidine addition, and centrifugation; Lane 3 is the supernatant of the 4 mM hexammine cobalt precipitation; and Lane 4 is the RNA pelleted in the hexammine cobalt precipitation but before any column separation.

FIG. 7 shows a FPLC chromatogram of V. proteolyticus RNA on a 25 mL high performance Q Sephamose anion exchange column (Pharmacia). The gradient ran over 12 column volumes from 0.30 M NaCl to 0.57 M NaCl in a column buffer of 20 mM bis-tris propane and 20 mM EDTA at pH 6.9. (see Example 9)

FIG. 8 shows a FPLC chromatogram of pCP3X3 aRNA-containing E. coli strain JM109 on a 25 mL high performance Q Sepharose anion-exchange column (Pharmacia). The gradient is run over 12 column volumes from .37 M NaCl to .57 M NaCl in a column buffer of 20 mM bis-tris propane and 20 mM EDTA at pH 6.9. (see Example 10)

FIG. 9 shows a FPLC chromatogram of selective precipitation purified β ribozyme on a 25 mL high performance Q Sepharose anion exchange column (Pharmacia). The gradient is run over 12 column volumes from .37 M NaCl to .7 M NaCl in a column buffer of 10 mM bis-tris propane and 20 mM EDTA at pH 6.9. (see Example 11)

FIG. 10 shows schematically a kit for convenient practice of the invention.

FIG. 11-13 Show light scattering-monitored compaction precipitations at 20° C. of 10 μg/mL nucleic acid in 10 mM bis tris propane buffer at pH 7.0. FIG. 11 (Top): plasmid DNA (pCMV sport β gal) with various compaction agents, FIG 12: (Middle) salmon sperm DNA with various compaction agents, FIG. 13 (Bottom) Vibrio proteolyticus total RNA with various compaction agents (spermidine was omitted from the Vibrio proteolyticus total RNA plot as condensation does not occur up to 700 charge equivalents.).

FIG.14. Ethidium bromide-stained 3% agarose gel showing Vibrio proteolyticus RNA fractionation by hexammine cobalt precipitation. Lane 1 is the BPER/spermidine initial lysate, Lane 2 is the supernatant of the 2 mM hexamine cobalt RNA precipitation (containing low molecular weight RNA), and Lane 3 is the resuspended and compaction agent-stripped pellet of the hexaminie cobalt precipitation (containing mainly 23 and 16S rRNA).

FIG. 15. PAGE 4%/10% composite gel stained SYBR Gold showing total Vibrio proteolyticus RNA separation by hexammine cobalt precipitation. Lane 1 is the BPER/spermidine initial lysate, and Lane 2 is the resusended and compaction agent stripped pellet of the 3.5 mM hexammine cobalt precipitation, showing that all species are precipitated and resuspended by this procedure.

FIG. 16. Ethidium bromide-stained 3% agarose gel showing the separation of pCP3X3 artificial RNA by hexamine cobalt fractionation. Lane 1 is the supernatant of the 2 mM hexamine cobalt RNA precipitation enriched in low molecular weight species, and Lane 2 is the resuspended and compaction agent stripped pellet of the 2 mM hexamine cobalt precipitation, containing primarily high molecular weight RNA.

FIG.17. SYBR Gold-stained 2% agarose gel showing the β ribozyme compaction precipitation protocol. Lane 1 is the supernatant of the first compaction precipitation (with 2 mM hexammine cobalt) and Lane 2 is the pellet of the first precipitation.

FIG.18. Multiple FPLC chromatograms from nondenaturing anion-exchange chromatography of RNA. Top: Chromatogram of Vibro proteolyticus RNA on a 10 ml high performance Q Sepharose anion-exchange column (Pharmacia). The gradient was run over 12 column volumes from 0.30 M NaCl to 0.45 M NaCl and over 20 CV's from 0.45 M NaCl to 0.57 M NaCl in a column buffer of 20 mM bis-tris propane and 20 mM EDTA at pH 6.9. Bottom: same as A except the aRNA pCP3X3 expressed in Escherichia coli JM109 was purified and the gradient was linear over 32 column volumes from 0.30 M NaCl to 0.57 M NaCl.

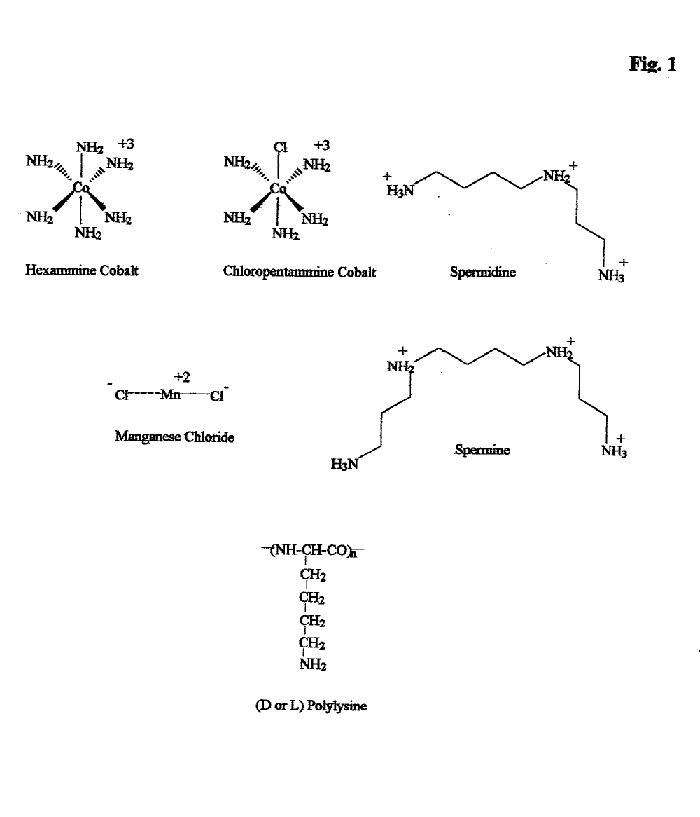

Table A gives preferred, more preferred, and most preferred levels of some of the parameters of the invention.

### DESCRIPTION OF THE PREFERRED EMBODIMENTS

### Example 1

### Large-scale Plasmid Preparation

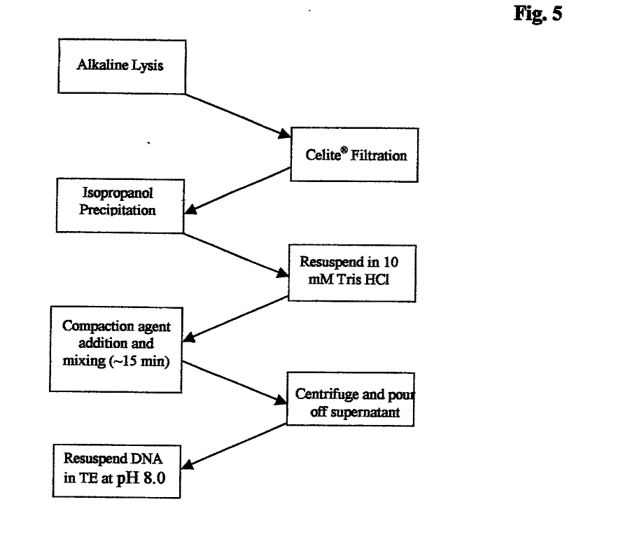

Referring to FIG. 5, E. coli JM109 strain containing pBGS19luxwt plasmid grown in Pseudomonas Media 187 (per liter of media add 10 g tryptone, 10 g yeast extra, 5 g K₂PO₄, 10 g glycerol, 5 mL salts solution to 1 L of distilled water where the salts solution contains 4.0 g MgSO₄*7H₂O, 0.2 g NaCl, 0.4 g FeSO₄*7H₂O, and 0.2 gMnSO₄*4H₂O in 100 mL of H₂O) at 37° C. in a 20 L Applikon fermentor (20 liter in-situ sterilizable bioreactor model number Z611120001). Overall fermenation time continues for about 12 hours and the cells grow to an OD₆₀₀ of about 20. The fermentor is harvested and the cells are pelleted at 4000 rpm in a Beckman centrifuge (6 L capacity rotor) for 30 minutes. Then the resulting pellets are optionally placed into plastic bags and heat-sealed to make crisps. The yield of the fermentation is approximately 440 g of wet cell paste.

Cells are lysed using a scaled-up version of the alkaline lysis procedure. First add 15 mL/gram wet cells of solution 1 (25 mM Tris Free Base, 10 mM EDTA, 50 mM Dextrose) and vortex. Next is added 15 mL/gram wet cells of Soluton 2 (1% SDS and 0.2 N NaOH) and the mixture is inverted 2-3 times and put on ice for 5 minutes (being careful at this point because the nucleic acids are extremely shear sensitive at high pH). Finally, we add 15 mL/gram wet cells of solution 3 (which is 600 mL of 5 M KAc, 115 mL of glacial acetic acid, and 285 mL of distilled water per liter) and invert 3-4 times and put on ice again for 5 minutes. The alkaline lysis not only disrupts the cells allowing DNA into solution but also most of the cellular proteins and chromosomal DNA are precipitated. At this point a white slime (mainly cell walls, precipitated protein, and precipitated chromosomal DNA) remains dispersed in the liquid.

At this point, a filtration is run to remove the cellular waste from the lysis step. 30 g/L Celite® Hyflo, a diatomaceous earth filter aid, are added to the product of the alkaline lysis and mixed with a plastic rod (If back pressure turns out to be a problemi the amout of Celite can be raised to 50 g/L). The suspension is then filtered through Whatman#1 filter paper in a 12-cm plastic Buchner funnel. Next, the DNA is precipitated by adding 0.7 volume -20° C. isopropanol to the filtrate and centrifuging in 250 mL bottles at 15,000× g in a Beckman model J2-21 centrifuge for 10 minutes at 4° C. Pellets are allowed to dry by inversion for 10 minutes and each is resuspended in low ionic strenght buffer (75 mL of 10 mM Tris buffer pH 8.0) An equal volume of 2.9 mM spermidine (spermidine trihydrochloride crystalline salt from Sigma Chemical, product number S 2501) solution in 10 mM Tris buffer pH 8.0 is added, the solution is mixed gently for 15 minutes at room temperature, and then centrifuge at 15,000× g for 10minutes at 24° C. The supernatant is discarded, 25 mL of wash solution (50% isopropanol with 300 mM NaCl, 10 mM MgCl₂, and 25 mM EDTA) is added to the tube containing the pelleted DNA, and this solution is incubated for 15 minutes at room temperature before a final centrifugation at 15,000× g for 10 minutes at 4° C. The supernatant is discarded, the nucleic acids pelleted with 70% ethanol (to eliminate any residual salts) and then each pellet is resuspended in 10 mL of TE (10 mM Tris HCl, 1 mM EDTA, pH 8.0) with 570 mM NaCl.

The plasmid is loaded onto a Spectrum FPLC column (2.5 cm×60 cm) packed with 150 mL Q Sepharose high performance anion exchange matrix and equilibrated in 10 column volumes of TE with 570 mM NaCl using a Pharmacia Automated FPLC system (Pharmacia Code number 18-1040-00). Loading and elution are perfomed at a linear velocity of 90 cm/hr. The column is washed with 1 column volume of TE with 570 mM NaCl followed by 4 column volumes of TE with 600 mM NaCl. A linear gradient of NaCl (600 mM to 700 mM NaCl) in TE over 4 column volumes is used to elute the DNA. Absorbance is monitored at 254 nm and appropriate fractions are collected with a final yield of 6.5±0.1 mg/6 grams dry cell weight. In other experiments, the yield is increased significantly by performing a temperature shift from 37 to 42° C. in the mid log phase of growth during the initial fermentation.

### Example 2

### Plasmid Mini-prep

Three mL of LB (1 liter contains 10 g of tryptone, 5g of yeast extract and 10 g of NaCl) medium containing 50 μg/mL kanamycin is inoculated with E. coli JM109 containing the plasmid pBGS19luxwt and grown overnight at 37° C. A 2 mL aliquot of this pipetted into a 2 mL microcentrifuge tube and then centrifuge at 14,000× g for 5 minutes to pellet the cells. The cells are then resuspended and lysed by the alkaline lysis method. (see reference 10) 300μl of solution 1 (25 mM Tris Free Base, 10 mM EDTA, 50 mM Dextrose) is added to the pellet and the pellet is resuspended by vortexing. After 300 μl of solution 2 (1% sodium dodecyl sulfate (SDS) and 0.2 N NaOH) are added and the mixture is inverted 3-4 times and placed on ice for 1-2 minutes.

Next 300 μl of ice-cold solution 3 (which is 600mL of 5 M KAc, 115 mL of glacial acetic acid, and 285 mL of distilled water per liter.) is added and the mixture is inverted 3-4 times and again placed on ice for 1 minute. Then the solution is centrifuged in a tabletop Eppendorf centrifuge at maximum speed and the supernatant is poured off to a new tube. The resulting solution is precipitated with 0.7 volume of −20° C. isopropanol. The pellet is resuspended in 500 μl 10 mM Tris HCl at pH 8.0 and 500 μl of 2.9 mM spermidine (Spermidine tihydrochloride crystalline salt from Sigma Chemical product number S 2501) stock is added. The tube is vortexed 10 seconds, incubated for 1 minute and centrifuged at 14,000× g for 2 minutes. The supernatant is discarded and 400 μl of wash solution (50% isopropanol with 300 mM NaCl, 10 mM MgCl2, and 25 mM EDTA) is added. The tube is again vortexed, incubated for 1 minute, and centrifuged at 14000× g for 3 minutes. The resulting pellet is washed with 70% ethanol and resuspended in 30 μl deionized H₂0.

### Example 3

### Selective Precipitation

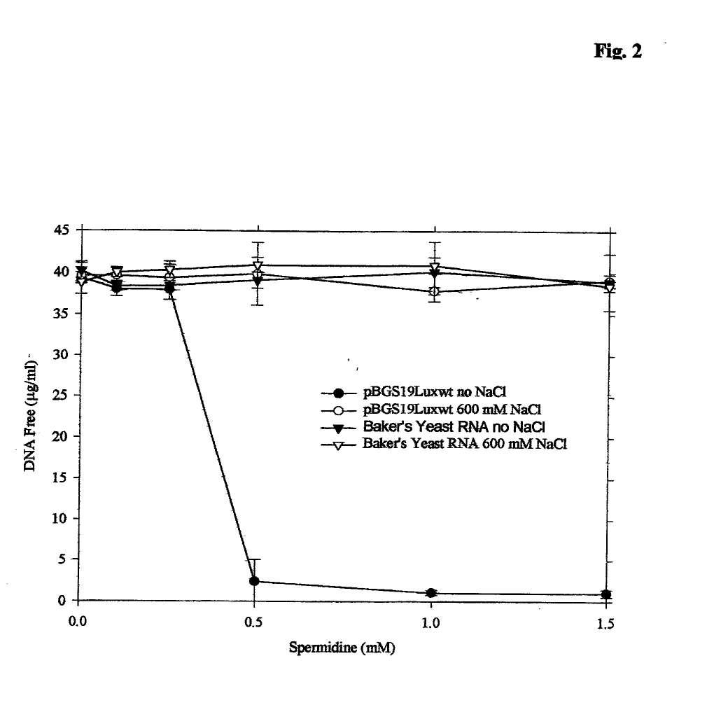

The concept of selective compaction precipitaton is demonstrated by using salmon sperm DNA, pBGS19luxwt (a 6 kB derivative of pUC19 expressing Vibrio harveyi luciferase), and total baker's yeast RNA. Both salmon sperm DNA (not shown) and the plasmid are efficiently precipitated with 0.5 mM spermidine at low ionic strength, but not in 600 mM NaCl. Yeast RNA, in contrast, does not precipitate at either ionic strength, as shown in FIG. 2. As practical applications will usually involve at least a modest ionic strength, the concentration spermidine required to precipitate plasmid DNA in the presence of 100 mM NaCl is measured and found to be 5-10 mM spermidine.

### Example 4

### Tetravalent Spermine

In other experiments conducted according to Example 3, plasmid DNA is precipitated in the presence of up to 200 mM NaCl substituting 10 mM of the (more potent) tetravalent spermine for spermidine. However, the spermine has two major draw backs: it is not as selective for DNA over RNA as spermidine so some RNA contamination can be present and spermine is difficult to completely remove from nucleic acids and will interfere with some later applications such as restriction enzyme digestion. Spermidine does not have these problem, thus it is our most preferred compaction agent for DNA applications.

### Example 5.

### Gram-scale Non-chromatographic Purification

Referring to FIG. 2 compaction precipitation used in a gram-scale non-chromatographic separation of plasmid DNA using the following steps: alkaline lysis (see reference 10), Celite filtration (see reference 11), isopropanol precipitation, LiCl precipitation (this step is optional), (see reference 12), isopropanol precipitation, compaction precipitation, and (if desired to remove compaction agents) washing with isopropanol/metal ion solution. In this procedure, the primary contribution of compaction precipitation is to remove the great majority of the RNA without the use of RNAse.

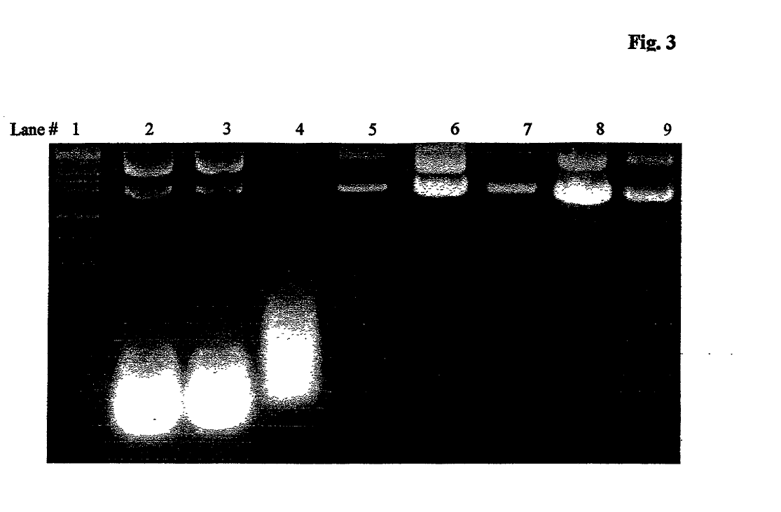

To eliminate compaction agent from the DNA pellet, several washing conditions have been examined. Preferably, a 50% isopropanol solution with 300 mM NaCl, 10 mM MgCl₂ and 25 mM EDTA is used to remove spermidine. Removal of compaction agents can also employ non-alcoholic solutions of high ionic strength, and may be unnecessary for plasmids, which are to be formulated with spermine or spermidine for pharmaceutical delivery purposes. The selectivity of precipitation can be seen in FIG. 3, which illustrates the stages of a typical compaction based plasmid purification. Lane 4 of FIG. 3 shows the supernatant from compaction precipitation, while Lane 5 shows the resuspended pellet from the same precipitation and Lane 6, a 10-fold overload of the plasmid pellet in which only a small amount of RNA can be visulalized. The, compaction precipitation increases the percentage of DNA in the sample from approximately 2% to approximately 99%.

### Example 7

### Effect of Compaction Precipitation on Subsequent Ion-exchange Polishing

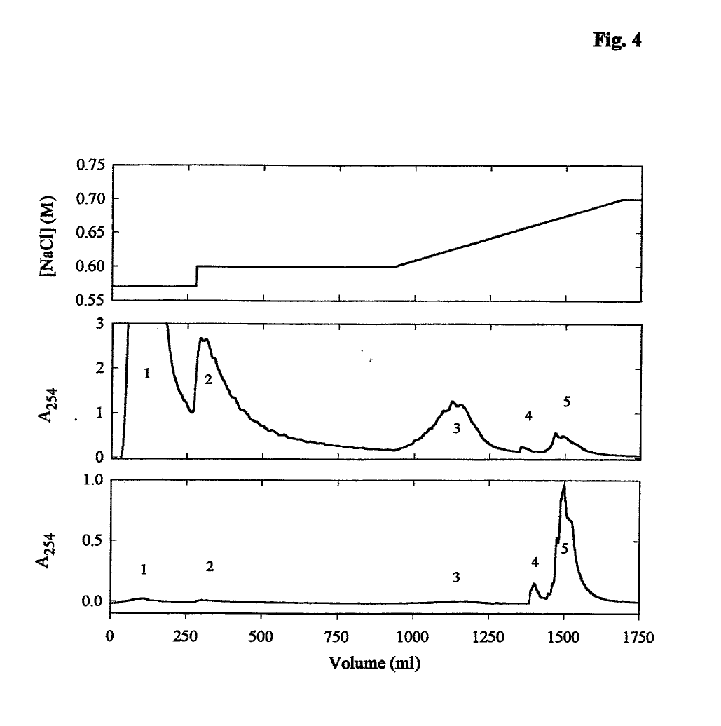

Referring to FIGS. 3 and 4, anion-exchange chromatography is commonly used for final purification of plasmid DNA (see reference 13). It is found that RNA removal improves the throughput of subsequent ion-exchange columns for plasid DNA reducing the resolution required to produce RNA-free plasmid. Anion-excange chromatography is performed on a Pharmacia FPLC System to eliminate residual traces of RNA (Figure 4). The selectively-precipitated plasmid, (10 mg plus the residal amount of RNA) resuspended in column running buffer and fractionated on a 150 ml Q Sepharose high performance anion-exchange column with the NaCl elution profile shown in FIG. 4 (top panel). The absorbance profile shown in the middle panel is the anio-exchange separation of resuspended isopropanol pellet not previously subjected to compaction precipitation, while the lower trace is the separation of material from which most RNA had been removed by a preliminary compaction precipitation step. The first two peaks are RNA passing through the column; during the initial 570 mM NaCl wash and an additional spike due to a step to 600 mM NaCl. The next peak (3) is a large RNA fragment, and the next two peaks are linear (4) and closed-circular plasmid (5) respectively, as determined by agarose gel electrophoresis FIG. 3, lanes 7 and 8).

After compaction precipitation, the amount of RNA to be removed is greatly reduced, the loading capacity for plasmid DNA; is higher (because of the lack of competing RNA) and the initial wash can be reduced in duration since very little RNA needs to be removed.

### Example 8.

### Small-scale Preparation of Plasmid DNA: “Mini-prep”

In addition to larger-scale pharmaceutical manufacturing, plasmid DNA is often purified on a smaller scale sequencing and other purposes. With this in mind, another embodiment of the invention is a mini-prep protocol based on compaction precipitation, which is directly scaled down from large-scale protocol.

The detailed protocol is as follows:

1. Grow plasmid containing LB cell cultures overnight at 37° C. with proper agitation

2. Centrifuge 2 mL of at 14,200× g for 5 minutes and decant supernatant

3. Resuspend cell pellet in 300 μl of GTE solution (50 mM glucose, 25 mM Tris-HCl (pH 8.0), 10 mM EDTA (pH 8.0))

4. Add 300 μl of Alkaline Lysis solution (0.2 N NaOH and 1% SDS) and gently invert 3-4 times. Store on ice for 1-2 minutes.

5. Add 300 μl of neutralization solution (60ml of 5 M KAc, 11.5 mL of glacial acetic acid, and 28.5 mL of distilled water per 100 mL of solution. Make sure to store at −20° C.) and allow it to sit for 1 minutes on ice.

6. Centrifuge at 14,200× g for 5 minutes and transfer supernatant to a new tube.

7. Add 0.7 volume of −20° C. isopropanol (0.84 mL), vortex and centrifuge at 14,200×g for 3 minutes

8. Decant supernatant and resuspend pellet in 400 μl of 10 mM Tris at pH 8.0.

9. Add 400 μl of 2.9 mM spermidine, vortex, incubate for 1 minute, and centrifuge at 14,200× g for 2 minutes.

10. Decant the supernatant

11. Wash the pellet with 800 μlof a fresh 50% IPA stock with 10 mM MgCl₂, 300 mM NaCl, and 25 mM EDTA . (I make up a stock of 20 mM MgCl₂, 600 mM NaCl, and 50 mM EDTA and add 1volume of IPA before I do the preps. Beware that over the course of 2-3 hours the metal ions will precipitate from the washing solution so mix fresh solution as needed (Optionally, a new stripping solution has been developed that consists of 50% EtOH, 300 mM NaCl and 10 mM EDTA which works well for this application without the issues with the precipitation of salts). Incubate for 1 minute and centrifuge for 2 minutes at 14,200× g.

12. Decant off wash solution.

13. Add 400 μl of 70% ethanol to wash the pellet. Preferably spin down the pellet for 20-30 seconds before decanting to make sure the pellet is not lost.

14. Resuspend in buffer of choice.

The final product PCR-is sequenced successfully on an ABI model 377 sequencer, yielding approximately 600 bases of usable sequence information, and well digested by restriction enzymes EcoR I and Hind III.

### Example 9

### Separation of bacterial RNA

With the proper selective precipitation strategy and the proper gradient as we have developed means of fast purification for bacterial rRNA.

Cells are grown in LB medium (10 grams of tryptone, 5 grams of yeast extract and 10 grams of NaCl per liter of media) in 1liter baffled shake flasks and the cultures are harvested in the mid-log phase(OD₆₀₀ 1.5 or less). Cells are the pelleted an stored at −80° C. until needed. Initial experiments are done on the wild type cell strain V. proteolyticus (see reference 29).

A non-ionic detergent mixture (BPER®) is used to lyse bacterial cultures. 60mL of BPER® per liter of cells at OD₆₀₀=1 and is found effective in cell lysis. To these lysed cells 1 volume of 5 mM spermidine HCl buffered in 20 mM bis-tris propane (BTP) at pH 6.9 is added to the lysate to precipitate unwanted chromosomal and plasmid DNA. The initial lysis is helped by the addition of spermidine, which is also an anti-bacterial agent (see reference 34).

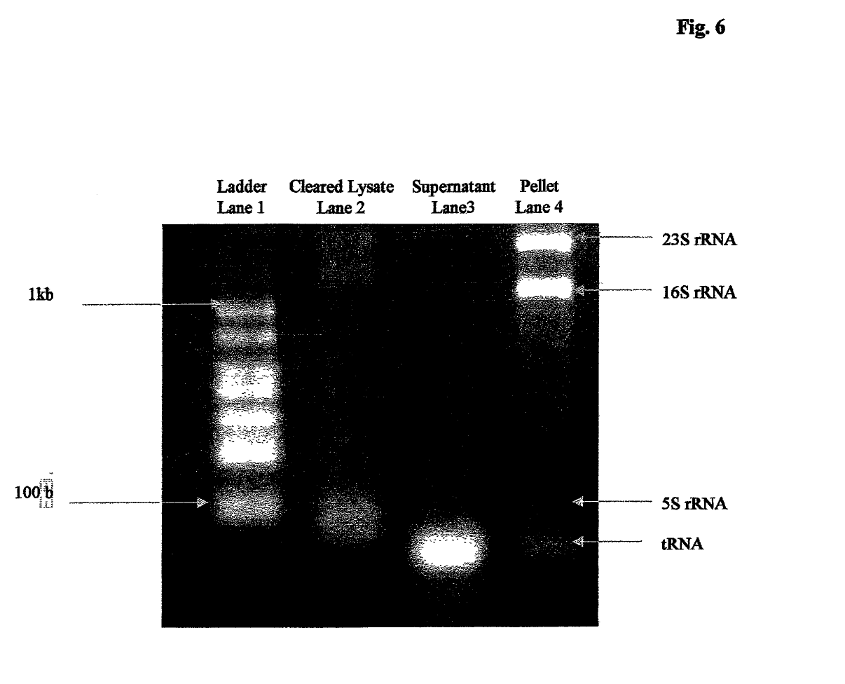

This mixture is then centrifuged and the supernatant is poured off into a new tube for further purification (Optionally, the BPER and spermidine solutions can be premixed into a lysis/DNA removal step). To the clarified lysate 4 mM hexammine cobalt was added and vortexed for 1 minute then centrifuged and the supernatant was discarded To remove hexammine cobalt from the RNA backbone, 50 mL of a 600 mM NaCl, 20 mM MgCl₂, and 50 mM EDTA buffered in 20 mM BTP at pH 6.9 was added. This solution is mixed for 2 mintes or until the pellet had redissolved. Next 2 volumes of ice cold EtOH are added to precipitate the RNA. Finally the RNA pellet is resuspended in 300 mM NaCl buffered in 10 mM BTP with 2 mM EDTA at pH 6.9 (column loading buffer). FIG. 6 is a 3% biogel (agarose) electrophoretic gel showing the separation after initial Lysis and the supermatant and stripped pellet from the above detailed separation.

The RNA is loaded, using a Pharmacia FPLC system, onto an Amicon FPLC column (2 cm×8 cm) packed with 25 mL Q Sepharose high performance media and equilibrated in 10 column volumes of column buffer (20 mM bis-tris propane and 20 mM EDTA at pH 6.9). Loading and elution are performed at linear velocity of 90 cm/hr. The column is washed with 4 column volumes of column loading buffer. RNA is eluted with a linear gradient of NaCl (300 mM to 570 mM NaCl in column buffer) performed over 10 column volumes. Absorbance is monitored at 254 nm and appropriate fractions are collected.

Nondenaturing anion-exchange chromatography can then be used to cleanup and separate each component of the rRNA fractions.

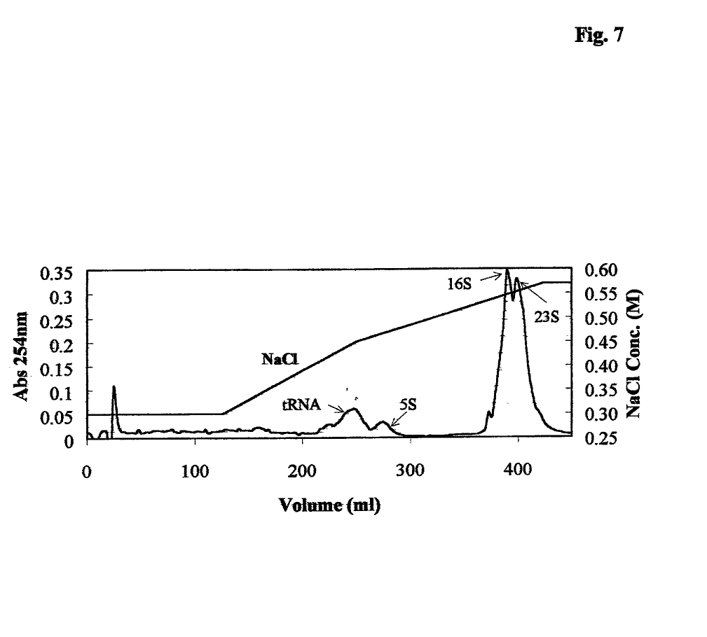

The anion-exchange columns use a high performance Q Sepharose strong anion exchanger from Pharmacia. FIG. 7 shows the absorbance profile obtained from a separation of V. proteolyticus RNA over the column. The column was loaded with selectively precipitated RNA enriched in rRNA. This allows the anion-exchange column to resolve 5S rRNA from tRNA. This separation is very difficult unless the amount of tRNA is reduced before the anion exchange column is run. Peaks 3 annd 4 are the 16S and 23S rRNA respectively. It is also possible to resolve the 16S and 23S rRNA on a nondenatured anion-exchange column as shown in FIG. 7 in the last two peaks.

### Example 10

### Separation of artificial stable RNA

Artificial stable RNA (see references 20-22,30) can be separated using the basic steps of Example 8 but with a few modifications. The aRNA pCP3X3 was produced in the E. coli JM109 and grown to an OD₆₀₀ from<1.5 in common LB media. Precipitation conditions and the procedure are identical to example 9 except for the anion-exchange column procedure. The anion-exchange column gradient is run between 0.30 M NaCl and 0.60 M NaCl all in column buffer consisting of 20 mM bis-tris propane and 20 mM EDTA at pH 6.9 over 10 column volumes. The plot of 254 nm absorbance vs. volume from the FPLC system for this purification is shown in FIG. 7.

### Example 11

### Separation of a bacterially-expressed ribozyme

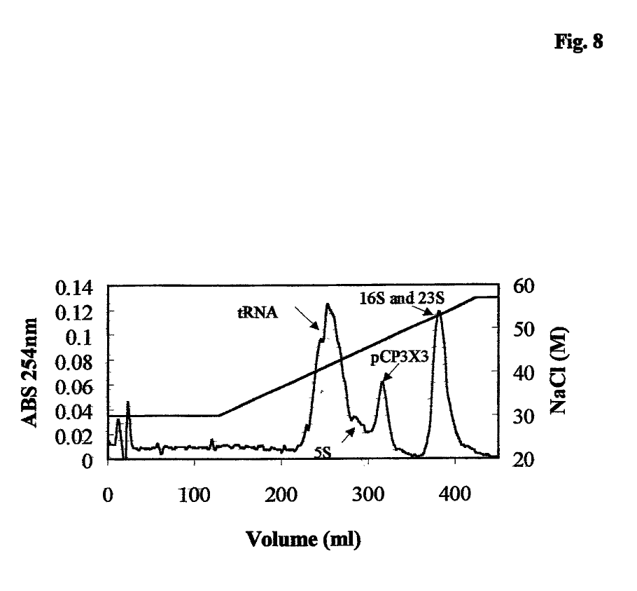

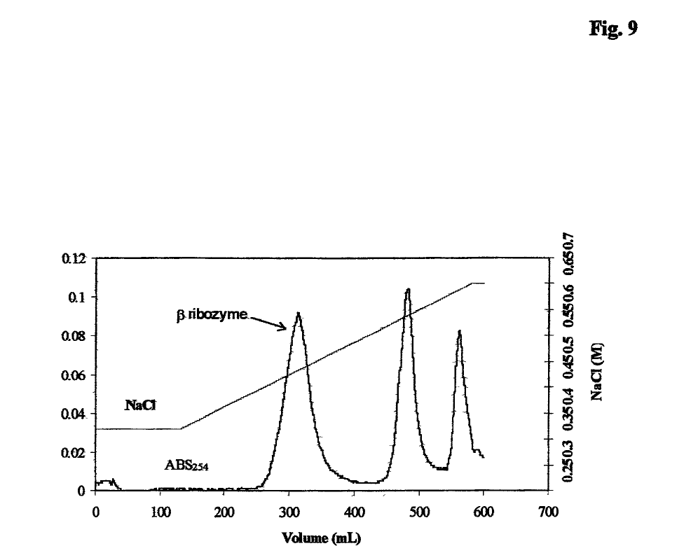

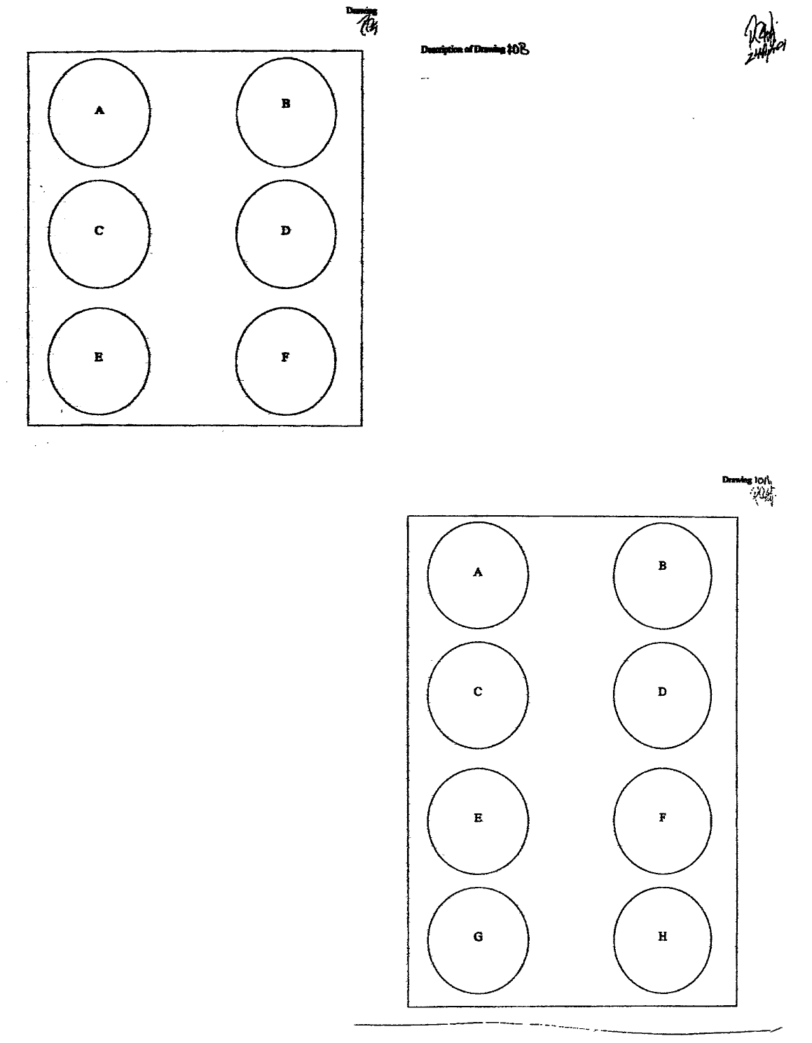

Ribozyme is produced using a T7-promoted plamid. β ribozyme was produced in strain MPD92 containing the T7 promoter-based plasmid pMPD4. (reference 23) Expression of β ribozyme was induced by adding 1 mM ITPG of at OD >0.4. All precipitation and lysis conditions are the same as example 9 but the anion exchange column is run slightly differently. The column running buffer for this separation is 10 mM bis-tris propane with 2 mM EDTA at pH 6.9 (done to spreadout the gradient.) The column is run from 0.3 M NaCl in column buffer to 0.65 M NaCl. The 254 nm absorbance vs. volume ploblem is shown in FIG. 8 and peak 1 corresponds to the β ribozyme. The problem with this separation is that the β ribozyme is 80 bases in length and cannot be resolved from tRNA and mRNA on an anion-exchange column as shown in FIG. 9. Alternative separation steps that can be tried are separation by size exclusion or hydroxyapatite chromatography (see references 31-33).

### Example 12

### RNA mini-prep

A RNA mini-prep is done with roughly the same concentrations of reagents detailed in Example 9 except on a much smaller scale, according to the following procedure. Many applications and variations to this mini-prep will be apparent to those skilled in the art. For instance, it can be done to produce total RNA and fractions of RNA enriched based on the size and amount of structure (double strandedness) of the RNA.

Protocol:

1. Grow cells and harvest in mid tog phase. (Maximizes RNA content) Centrifuge at Max speed in a table top centrifuge for 5 minutes and decant supernatant (store at −80° C. if not used immediately)

3. Add 150 μl (15 mL/4 grams of wet cells) of BPER (Pierce, 78248) and resuspend pelleted cells by vortexing.

4. Incubate at room temperature for 2 minutes.

5. Add 150 μl of 2.9 mM spermidine HCl (Sigma, S-2501) buffered in 20 mM bis-tris Propane (BTP) at pH 6.9, votex and incubate for 5 minutes.

6. Centrifuge at 12,000 rpm 10 minutes at 4° C.

7. Decant supernatant to a new tube and add 300 μl of 4 mM Co(NH₃)₆ buffered in 20 mM BTP (Sigma, H-7891, vortex, and incubate for 5 minutes. (for total RNA use 7 mM Co(NR₃)₆ and for 16S and 23S rRNA use 2.5 mM Co(NH₃)₆)

8. Centifuge at 12,000 rpm for 10 minutes at 4° C.

9. Decant supernatant resuspend in 300 μl mL of stripping solution (600 mM NaCl, 10 mM MgCl₂, and 25 mM EDTA buffered in 20 mM BTP at pH 6.9 (all chemicals from Sigma)), vortex and incubate at room temperature for 3-5 minutes.

10. Add two volumes of ice-cold ethanol, vortex and, centrifuge at 10,000 rpm at room temperature for 5 minutes.

11. Decant supernatant and resuspend in buffer of choice.

### Example 13

### Assay by compaction precipitated probe target hybrids of 5S rRNA with fluorescein labeled oligonucleotides

The production of 5S rRNA accomplished according to the protocol detailed in Example 12. The modification to the procedure of Example 12 occurs after the addition of 5 mM spermidine and before the addition of 4 mM hexammine cobalt. After step 6 in Example 12 and after the supernatant is added to a new tube ˜10 nmols of 5′ fluorescein labeled probe (5′-TGC-CTG-GCG-ACC-ATA-GCG-ATT-T-3′) is added. This solution is then heated to 90° C. for 30 seconds and then rapidly cooled on ice. Then are carried out the rest of the steps in Example 13 but except resuspend in 300 μl of distilled H₂O in step 11. Next, using a microplate fluorometer with the probe will not bind and another fluorescence is read in comparison with controls (e.g. same hybridization protocol with a strain of cell for which the probe will not bind and another without the labeled probe). If the correct target sequence is present the fluorescein emission will be well above background.

### Example 14

### Clarification of protein-containing solutions

This example demonstrates (see reference 35) how DNA can be removed from lysates to aid in protein purification. First cells were grown in the Applikon fermenter (as in example 1) and the cells were an E. coli cell strain 1547 (a derivatve of JM109) Approximately 120 grams of wet cells were resuspended in 20mM HEPES buffer+0.1% TritonX-100 at pH 8.0. Then the lysate is run through a French cell press twice to lyse cells. After lysis, 6 mL of 0.5 M spermidine HCl solution is added and the overall pH was readjusted to 8.0. Next the lysate is spun down at 12,000× g in a Beckman J2-21 centrifuge at 4° C. This cleared lysate is run over a 300 mL High performance Q sepharose column at a flow rate of10 mL/minute and an optimized gradient for proteins eluted. After spermine precipitation the lysates are visibly less viscous, have a negligible amount of nucleic acid remaining as checked using agarose gel electrophoreses and protein concentrations are identical to that of the untreated solution as determined by BioRad's Protein Assay (a Bradford Assay).

### Example 15

### Mini-prep from difficult host strains

The technique of Examples 1 and 2 are applied to host strains that are difficult to separate nucleic acids from, in this example, the strain of Pseudomonas LD2, which has a polysaccharide coat on its outer membrane. This cell strain is extremely hard to process using conventional technology since the polysaccharides will co-purify with the plasmid DNA, chromosomal DNA, etc. The selective precipitaton done according to Examples 1 and 2 is an extremely effective separation on both the large and small scale for these hard to purify host strains. The protocols in Examples 9 and 12 can also be applied to purify RNA from these same hard to purify strains.

### Example 16

### Isolation of nucleic acid-binding proteins

This example demonstrates the use of compaction precipitation to produce an enriched sample of a nucleic-acid-binding protein, (this protein is a DNA-binding repressor which binds to a lac repressor found in the plasmid). E. coli cells horboring plasmid encoding a protein with affinity for a DNA sequence found in the plasmid were grown in the Applikon fermenter (as in example 1). Approximately 120 grams of wet cells were resuspended in 20 mM HEPES buffer+01% Triton X-100 at pH 8.0. and the lysate is run through a French cell press twice to lyse cells. Next the lysate is spun down at 12,000× g in a Beckman J2-21 centrifuge at 4° C. After centrifugation, 6 mL of 0.5 M spermidine HCL solution is added to the supernatant and the overall pH is readjusted to 8.0. Next the precipate is spun down at 12,000× g in a Beckman J2-21 centrifuge at 4° C. Resuspension of the pellet resulting from this centrifugation results in a solution enriched in the DNA-binding protein.

### Example 17

### Separation of natural plasmids for quick recognition of degradative pathways

The process of Examples 1 and 2 is applied to the separation of natural plasmids from pseudomonas cells, which, encode for an aromatic degradative pathway. The isolated plasmids are used in in efficiently searching for the genes encoding the degradative pathway.

### Example 18.

### Large-scale (Low Endotoxin) Plasmid Preparation

In other experiments conducted according to Example 1, the resuspended plasmid product is found by the Pyrochrome(R) (Chromagenic Formulation) Limulus Amebocyte Lysate (LAL) assay (Associates of Cape Cod, Inc.) to contain less than 0.3 Unis endotoxin per microgram plasmid (EU/μg or IE/μg).

### Example 20

### Additional Washing

Additional washing steps are can be added to Example 1 such that the end sample, contains less than 0.1 Units endotoxin per microgram plasmid. 70% EtOH or a 1.5 mM spermidine rinse after inital pelleting by compaction precipiatation is used as a washing step for plasmid during the process.

Washing can be done by difiltration, especially on a larger scale, and can be as important as centrifugation, for some applications.

### Example 21

### Multiple Compaction precipitations

Example 1 is can be augmented by performing main process of compaction agent precipitation multiple times in series to provide plasmid containing less than 0.1 Units endotoxin per microgram plasmid. Also, reduced levels of other contaminants (e.g. RNAse, RNA, proteins, DNAse) are obtained possible with multiple compaction precipitations.

### Example 22

### Tetravalent Spermine (Low Endotoxin)

In other experiments conducted according to the process of Example 3, plasmid DNA is precipitated in the presence of up to 200 mM NaCl by substituting 10 mM of the (more potent) tetravalent spermine for spermidine.

The resuspended plasmid product is found by the Pyrochrome(R) (Chromagenic Formulation) Limulus Amebocyte Lysate (LAL) assay (Associates of Cape Cod, Inc.) to contain less than 0.3 Units endotoxin per microgram plasmid DNA (EU/μg or IE/μg). Refined procedures or repeated precipitations provide product containing less than 0.1 Units endotoxin per microgram plasmid DNA (EU/μg or IE/μg).

Examples for Filtration based Preparations

### Example 23

### Large-scale Plasmid Separation using Filtration

E. coli JM109 strain containing pCMV sport β gal plasmid grown in Pseudomonas Media 187 (per liter of media add: 10 g tryptone, 10 g yeast extract, 5 g K₂HPO₄, 10 g glycerol, 5 mL salts solution to 1 Lof distilledwater where the salts solution contains 4.0 g MgSO₄*7H₂O, 0.2 g NaCl, 0.4 gFeSO₄*7H₂O, and 0.2 g MnSO₄*4H₂O in 100 mL of H₂O) at 37° C. in a 20 L Applikon fermentor (20 liter in-situ sterilzable bioreactor model number Z611120001). Overall fermentation continues for about 12 hours and the cells grow to anOD600 of about 20. The fermentor is harvested and the cels are pelleted at 4000 rpm in a Beckman centrifuge (6 L capacity rotor) for 30 minutes. Then the resulting pellets are optionally placed into plastic bags an heat-sealed ad frozen to make crisps. The yield of the fermentation is approximately 440g of wet cell paste. Cells are lysed using a scaled-up version of the alkaline lysis procedure. First add 15 ml/gram wet cells of solution 1 (25 mM Tris Free Base, 10 mM EDTA, 50 mM Dextrose) and vortex. Next is added 15 ml/gam wet cells of Solution 2 (1% SDS and 0.2 N NaOH) and the mixture is inverted 2-3 times and put on ice for 5 minutes (being careful at this point because the nucleic acids are extremely shear sensitive at high pH). Finally, we add 15 mL/gram wet cells of solution 3 (which is 600 mL of 5 M KAc, 115 mL of glacial acetic acid, and 285 mL of distilled water per liter) and invert 3-4 times and put on ice again for 5 minutes. The alkalinelysis not only disrupts the cells allowing DNA into solution but also most of the cellular proteins and chromosomal DNA are precipitated. At this point, a white slime (mainly cell walls, precipitated protein, and precipitated chromosomal DNA) remains dispersed in the liquid. A filtration is run to remove the cellular waste from the lysis step. 30 g/L Celite® Hyflo, a diatomaceous earth filter aid, is added to the product of the alkaline lysis and mixed with a plastic rod. The suspension is then filtered through Whatman ∩1 filter paper in a 12-cm plastic, Buchner funnel. Next, the DNA is precipitated by adding 0.7 volume −20° C. isopropanol (IPA) to the filtrate and centrifuging in 250 mL bottles at 15,000× g in a Beckman model J2-21 centrifuge for 10 minutes at 4° C. (an alternative to centrifuging is the use of filtraion to catch the IPA induced precipitant). Pellets are allowed to dry by inversion for 10 minutes and each is resuspended in low ionic strength buffer (75 mL of 10 mM Tris buffer pH 8.0). An equal volume of 2.9 mM spermidine (spermidine trihydrochloride crystalline salt from Sigma Chemical, produt number S 2501) solution in 10 mM Tris buffer pH 8.0 is added, the solution is mixed gently for 15 minutes at room temperature, and then filtered through a 0.45 mm. 25 mL of wash solution (50% EtOH with 300 mM NaCl, and 12.5 mM EDTA) is added to vacuum filter vessel and allowed to pass through a 0.45 mm. 25 mL of wash solution (50% EtOH with 300 mM NaCl, residual salts) is pressed over the filter twice (approximately 20 mLs total). Then approximately 10 mL of TE (10 mM Tris HCl, 1 mM EDTA, pH 8.0) is used to resuspend the purified plasmid DNA. The filter used in this experiement is a Corning Brand disposable vacuum filter with a 45 um cellulose acetate filter. These separations will also work with other filters as long as the filters have a negligible affinity for nucleic acids and that the said filters have an adequate pore size and structure to capture the nucleic acid of interest without having problems with the filters actually clogging. In the latter case, a filter aid that has little or no affinity for nucleic acds especially plasmid DNA in this case can be used to enhance the flow properties of the filter.

### Example 24

### Filtration-based Plasmid Mini-prep

Three mL of LB (1 liter contains 10 g of tryptone, 5 g of yeast extract and 10 g of NaCl) medium containing 50 μg/mL kanamycin is inoculated with E. coli JM109 containing the plasmid pBGS19luxwt and grown overnight at 37° C. A 2 mL aliquot of this culture is pipetted into a 2 mL microcentrifuge tube and then centrifuged at 14,000× g for 5 minutes to pellet the cells. The cells are then lysed by the alkaline lysis method. (see reference 10) 300 μl of solution 1 (25 mM Tris Free Base and 10 mM EDTA) is added to the pellet and the pellet is resuspended by vortexing.

After 300 μl of solution 2 (1% sodium dodecyl sulfate (SDS) and 0.2 N NaOH) are added and the mixture is inverted 3-4 times and placed on ice for 1-2 minutes. Next 300 μl of ice-cold solution 3 (which is 600 mL of 5 M KAc, 115 L of glacial acetic acid, and 285 mL of distilled water per liter.) is added and the mixture is inverted 3-4 times and again placed on ice for 1 minute. Then the solution is centrifuged in a tabletop Eppendorf centrifuge at maximum speed and the supernatant is poured off to a new tube. The resulting solution is precipitated with 0.7 volume of −20° C. isopropanol. Then the solution is run over a centrifuge filter column (by loading the column and centrifuging at max speed in the before mentioned Eppendorf centrifuge) to remove the IPA induced nucleic acid aggregate from solution. Then 250 μl of 1.45 mM spermidine in 10 mM Tris HCl at pH 8.0. (Spermidine trihydrochloride crystalline salt from Sigma Chemical product number S 2501) was run over the mini column (to wash away contaminants leaving highly purified plasmid DNA on the filter. 400 μl of wash solution (50% EtOH with 300 mM NaCl, and 12.5 mM EDTA) is put over the filter to remove the compaction agent. Then the filter is washed with 70% ethanol and finally (in a new tube) the plasmid DNA is resolublized with 30 μl deionized H₂O.

### Example 25

### RNA mini-prep

A RNA mini-prep is done with roughly the same concentrations of reagents detailed in Example 9 except on a much smaller scale, according to the following procedure Many applications and variations to this mini-prep will be apparent to those skilled in the art. For instance, it can be done to produce total RNA and fractions of RNA enriched based on the size and amount of structure (double strandedness) of the RNA.

Protocol:

1. Grow cells and harvest in mid-log phase. (Maximizes RNA content)

2. Centrifuge at Max speed in a table top centrifuge for 5 minutes and decant supernatant (store at −80° C. if not used immediately)

3. Add 150 μL (15mL/4 grams of wet cells) of BPER (Pierce, 78248) and 150 μL of 5 mM spermidine in 10 mM bis tris propane at pH 6.9, and resuspend pelleted cells by vortexing.

4. Incubate at room temperature for 2 minutes.

5. Centrifuge at 12,000 rpm for 10 minutes at 4° C.

7. Decant supernatant to a new tube and add 300 μL of 4 mM Co(NH3)6 buffered in 20 mM BTP (Sigma, H-7891), vortex, and incubate for 5 minutes. (for total RNA use 7 mM Co(NH3)6 and for 16S and 23S rRNA use 2.5 mM Co(NH3)6)

8. Apply solution from step 7 to a microfuge spin filter column and centrifuge until all of the RNA precipitant is captured in the filter.

9. Run 300 μL of stripping solution (50% EtOH, 300 mM NaCl, and 12.5 mM EDTA buffered in 20 mM BTP at pH 6.9 (all chemicald from Sigma)) over the microfuge column to strip the hexammine cobalt from the RNA

10. Next wash the filter with 70% EtOH by applying the spin filter column and centrifuging the EtOH solution through the column.

11. Snap the microfuge column into a new tube and resuspend the RNA on the filter with a buffer of choice and spin the fluid through the column to recover the RNA.

This filtration-based RNA separation protocol can also be scaled up to for larger-scale RNA production using vacuum based filters like the ones used in the Large-scale Plasmid Separation using Filtration Example 24, or using tagential-flow filters. Multiple samples can be processed in parallel using a microtiter plate-format multi-sample filtration block.

### Example 26

### Compaison of Compaction Agents

Using the protocol of Example 8 but with the spermidine concentration cut in half on all three plasmind tested works well. When overloaded (˜1 μg of the plasmid DNA per well) there is a slight signature of RNA but that is expected from solution transfer effects and the fact that the separation of the alkaline lysate from the white protein/chromosomal DNA floc is difficult to accomplish perfectly. Plasmid was produced with a 260/280 ratio of 1.86-1.91 within the 260/280 ratio range of quality plasmid DINA and a yield that included all of the plasmd DNA in the sample (comparing a control isopropyl alcohol (IPA) only run to the compaction runs the plasmid bands are of equal magnitude). It is also found that using the protocol where the IPA pellet is resuspended in the compaction agent containing solution directly there are obtained 260/280 ratios that vary from 1.92 to 2.00 and RNA is very visible on the 0.8% E-gels. Also, all of the spermidine lots Sigma (two sub-lots) and a lot from calbiochem worked equally well

### Example 27

### Separation of Chromosomal/Genomic DNA

Another possible kit based on compaction precipitation is for the separation of genomic DNA from both eukaryotes and prokaryotes. The preferred lysis method is using lysozyme, protease K, with some EDTA and nonionic detergents to aid in the destruction of the cell membrane. In addition, other lysis techniques may be useful with this technique if undamaged genomic DNA is released during the course of the procedure. Next, an IPA precipitation can be done to desalt the solution and the a compaction precipitation using a resuspension solution (≦2.9 mM spermidine trihydrochloride (from Sigma Chemical Co., product number 233994), a stripping solution (300 mM NaCl with 10 mM EDTA in 50% ethanol), and a final resuspension solution (preferably TE which is 10 mM Tris with 1 mM EDTA at pH 8.0).

### Example 28

### Microscale Separations ofNucleic Acids using Compaction Agents

Currently, there is large amount of attention being placed on micro-scale devices that are capable of PCR, sequencing mass spectrometry, chromatography, and etc. that fall under the general term Laboratories on a chip. These labs on a chip are usually based on the etching of silicon wafers and the microchip fabrication methods using in the semiconductor industry.

Compaction can be used on this scale for separating nucleic acids or in an assay format (e.g. detection of microorganisms, sequencing, separation of genomic DNA for genetic testing, etc.).

An example of such a device has etched fluidic channels on a surface through which a compaction agent containing stream and a sample stream can meet and a target nucleic acid can be precipitated. Using etched microfilters (small channels can be etched into the surface) the separations can be done by flowing solution based on the art taught in this patent application to perform separations for later processing. Also, the assay described in Example 13 can be applied in a similar micro-scale device.

### Examples 29 -33

### Structured RNA Isolation and Fractionation with Compaction Agents

The purification of RNA from bacterial cells has traditionally been achieved by phenol/chloroform extraction and polyacrylamide gel electrophoresis (1). These methods, however, require considerable time and labor for modest yields, and involove the use of toxic substances. Selective precipitation is a high-capacity purification method widely used in the isolation of proteins (2), (3). While mucleic acids also cna be purified using precipitation by alcohols, polyethyleneimine, and compaction agents (4), most precipitation methods lack selectivity among different nucleic acid types.

Compaction agents generally are small, cationic molecules, which bind in either the major or minor grooves of double-stranded mucleic acid molecules. Compaction agents change the conformation of mucleic acids through neutralization of the phosphate anion backbone and by the physical bridging of helices (5), (6). We have recently demonstrated the selective precipitation of plasmid DNA from Escherichia coli alkaline lysates using compaction agents (7).

In the present work, the extension of compaction precipitation to structured RNA isolation is described. Compaction precipitation drastically reduces the concentration of proteins and DNA, yeilding highly enriched RNA. Hexammine cobalt is particularly useful for this application, as it has a relatively high selectivity for RNA, particularly at polypurine sequences. (8).

In these Examples 29-33 , the selective precipitation and partial fractionation of RNA from cell lysates using compaction agents is detailed.

### Example 29

### Strains, Cultures, and Nucleic Acids

Bacteria are grown in LB medium in 1 liter baffled shake flasks, harvested in the mid-log phase (OD₆₀₀≦1.2), and cells pelleted and stored at −80° C. until needed. Initial experiments employ wild type Vibro. proteolyticus (9). The engineered 5S artificial RNA pCP3X3 (160 nt) was produced in Escherichia coli JM109 using the plasmid pCP3X3 (9), (10), (11). □ ribozyme (87 nt, recognizing the HIV type 1 integrase viral RNA) was produced in Escherichia coli strain MPD92 containing the T7 promoter-based plasmid pMPD4 (12) and induced with 1 mMITPG at OD=0.4.

Condensation experiments use salmon sperm DNA (sigma, average length 2 kb), plasmid DNA (7.9 kb pCMV sport □ gal originally obtained from Gibco, purified by comaction precipitation (7)), and V. proteolyticus Vibrio proteolyticus RNA purified by the total RNA protocol described below.

### Example 30

### Condensation Experiments

Condensation curves are used to determine selectivities of compaction agents for different nucleic acids. A SPEX Fluorolog-2 Fluorometer is used with L-format excitation and emission wavelengths set at 500 nm. To 3 mL of 10 μg/mL nucleic acid, compaction agents are added with constant stirring in a series of aliquots at 210 -second intervals until scattering intensity is constant. Lysis: A non-ionic detergent mixture, Bacterial Protein Extraction Reagent (BPER; Pierce), was mixed with an equal volume of 5 mM spermidine in 20 mM bis tris propane at pH 6.9, and this lysis mixure was used at 120 mL of lysis mix per liter of culture (OD₆₀₀= 1) for room temperature cell lysis. Lysis is allowed to proceed for one minute, then the mixture was centrifuged 10 minutes at 10,000× g, and the clarified supernatant decanted to a new centrifuge-tube. The effect of the spermidine is to precipitate unwanted chromosomal and plasmid DNA (7), and possibly also to enhance lysis (13).

### Example 31

### Initial Precipitation of rRNA

The clarified-lysate is mixed with an equal volume of 4 mM hexammine cobalt, vortexed for 1 minute, and centrifuged (10 minutes, 15,000× g at 4° C.). The resulting pellet (primarily rRNA) is then carefully washed with 70% ethanol. To strip hexammine cobalt from the RNA backbone the pellet is dissolved (100 mL per liter of orginal culture at OD₆₀₀=1) in 300 mM NaCl, 20 mM bis tris propane at pH 6.9, 20 mM EDTA (“nondenaturing column buffer” ), and (optionally) 6 M urea and incubated for at least 2 minutes. The resuspended RNA can then be purified by chromatography or precipitated by the addition of 2 volumes of ice-cold ethanol.

### Example 32

### Light Fraction Compaction Precipitation

A second hexammine cobalt precipitation is optionally performed to precipitate the smaller RNA fragments (mRNA, tRNA, ribozyme, etc.) and to reduce protein content of the final product. The supernatant of the initial hexammine cobalt precipitation is mixed with 0.33 volumes of 20 mM hexammine cobalt, vortexed for 1 minute, incubated with gentle mixing for 15 minutes at 4° C., and centrifuged (10 minutes, 15,000× g at 4° C.). The supernatant is then discarded and the low molecular weight-RNA pellet stripped as described above.

### Example 33

### Nondenaturing Anion-Exchange Chromatography:

The RNA resuspended in colume loading buffer after lysis and initial precipitation(s) is loaded onto an Amicon FPLC column (2 cm×8 cm) packed with 10 mL Q Sepharose high performance anion-exchange resin (Pharmacia) pre-equilibrated with 10 column volumes nondenaturing column buffer.

Loading and elution are performed at a linear velocity of 90 cm/hr using a Pharmacia FPLC system at 4° C. with absorbance monitoring at 254 nm. The column is washed with 12 column volumes of nondenaturing column buffer, and RNA was eluted with a linear gradient of 300 mM to 570 mM NaCl in nondenaturing column buffer over +column volumes.

### Example 33

### Small-Scale RNA Isolation:

The protocols described above can be directly scaled down for small-scale preparation of RNA. The 250 mL bacterial culture used above is scaled to 2 mL and all other volumes reduced proportionally. For small-scale stripping of compaction agents and alternative to the use of column loading buffer is to resuspend the RNA pellets in a stripping solution containing 600 mM NaCl, 50 mM EDTA, 20 mM MgCl₂ in 20 mM bis tris propane at pH 6.9. RNA is then precipitated with 2 volumes of anhydrous ethanol and resuspended in an appropiate buffer.

Modifications

Specific comositions, methods, or embodiments discussed are intended to be only illustrative of the invention disclosed by this specification. Variations on these compositions, methods, or embodiments are readily apparent to a person of skill in the art based upon the teachings of this specification and are therefore intended to be included as part of the invention disclosed herein.

For example, another potential application of selective precipitation is to the isolation of RNA; preliminary work indicates that potent compaction agents can not only precipitate RNA but also fractionate different size RNA molecules. Finally, compaction agent can be substituted for protamine, streptomycin, etc. in cleaning up cell lysates for purification of intracellular proteins. In the above Examples we have demonstrated that addition of a compaction agent can precipitate DNA or RNA form crude cell lysates, greatly reducing product viscosity and improving the performance of subsequent chromatographic columns, see e.g. Example 14.

Most preferably, the invention comprises a method of preparing substantially purified DNA, without the use of nucleases or proteases, (more preferably free of animal-derived proteins or free of non-host-derived ribonucleases), by adding an effective amount of a compaction agent to a lysate so as to precipitate from said lysate, DNA having a content of RNA of less than 3% by weight.

Using compaction precipitation, when a tagged probe (e.g. fluoresceinated probe) is added to a solution containing its target, a double stranded nucleic acid is formed and this new structured hybrid can be selectively precipitated while the single stranded probe will be left in solution.

A particularly preferred application of the protocols of the invention is for producing pharmaceutical grade plasmid DNA with an RNAse level, chromosomal DNA level, contaminating protein level, an endotoxin level and a RNA level below the guidelines set forward by the U.S. Food and Drug Administration, (See e.g. the FDA website at http://www.fda.gov).

Centrifugation is preferred to enhance the speed and usability of kits including those listed in Examples 1, 8, 23, 26 and 27.

Some preferred embodiments of the invention comprise:

A. A method of preparing substantially purified DNA, without the use of nucleases or proteases, by adding an effective amount of a compaction agent to a lysate to precipitate, from said lysate, DNA having a content of RNA of less than 3% by weight.

B. A method for the production of purified DNA having a content of RNA of less than about 3% by weight, comprising in combination the following steps:

A. lysing a cell mass to liberate the nucleic acids

B. optionally precipitating some additional moieties.

C. optionally adjusting the ionic strength and/or plasmid concentration and;

D. precipitating a substantial fraction of the DNA away from contaminating RNA and protein by addition of an effective amount of a compaction agent.

C. A composition of matter comprising DNA, substantially free of added nucleases, and containing less than about 3% by weight RNA

D. A method of treatment of a mixture comprising desired RNA product and contaminating DNA comprising mechanical lysis of the mixture in the presence of a comprising mechanical lysis of the mixture in the presence of a compaction agent to precipitate at least a portion of the contaminating DNA.

E. A composition of C above additionally comprising less than 0.0001 weight % RNAse.

F. A composition of claim 3 comprising a plasmid DNA encoding proteins for use as a vaccine.

G. A composition of claim 6 wherein the protein comprises influenza proteins.

H. A method according to claim 2 wherein DNA is separated from endotoxin to a level of less than 0.1 EU/μg plasmid DNA.

I. A method for making a biochemical assay comprising hybridizing a labeled probe to a target and thereafter precipitating the probe and the target, leaving the unhybridizing probe largely in solution.

J. A method for making an assay according to claim 9 wherein the labeled probe comprises a fluorescein-labeled oligonucleotide.

K. A method according to B above for producing ribosomal RNA, chromosomal DNA, plasmid DNA, aptamers, artificial RNA, or mRNA or other natural or synthetic nucleic acids.

L. The method of A above comprising producing plasmid having an undetectable content of ribonucleases by standard assays.

M. The composition of C above additionally comprising a content of eukaryotic ribonucleases of less than 0.1% by weight.

N. The method of A above comprising producing plasmid having a content of eukaryotic ribonucleases of less than 0.001% by weight.

O. The method of A above in which the addition of the compaction agent comprises the addition of two or more different mixed compaction agents whereby improved separation efficiency results.

P. The method of P above further comprising subsequent chromatographic column purificaton wherein prior use of compaction agents enhances the overall loading capacities of plasmid DNA on anion-exchange columns by elimination of the majority of contaminating RNA and other biomolecules, which would otherwise impair the subsequent chromatography.

Q. A method according to A above additionally comprising stripping the compaction agent by a stripping method selected from the group comprising high salt addition and/or a pH shift.

R. A composition for the recovery of DNA comprising a mixture of combined reagents, one of which lyses and one of which precipitates DNA to clarify a cell mass.

S. A composition according to R above in which the lysign agent comprises a nonionic detergent.

T. A method according to B above in which lysing cells is accomplished at a low salt concentration, which is applied to lyse RNA-containing cells.

U. A method according to B above wherein the method is applied to remove large nucleic acid molecules from low ionic strength bacterial lysates.

V. A method according to B above additionally comprising a technique selected from the group consisting of use of French cell press, addition of nonionic detergent, lysozyme addition, microfluidizer, freeze-thaw or any other relatively low ionic strenght lysis technique to produce nucleic acid free lysates for later protein recovery.

W. A method according to A above comprising simultaneous application of the method in parrallel mini-prep procedures for a plurality of cell masses.

X. A method of assay comprising precipitating a labeled probe while it is hybridized to a target.

Y. A method according to B above producing pharmaceutical grade plasmid DNA with an RNAse level, chromosomal DNA level, contaminating protein level, an endotoxin level and a RNA level below the guidelines set forward by the Food and Drug Agency at website: http://www.fda.org.

Z. A method according to B above additionally comprising a further separation step comprising one or more techniques selected from the group consisting of: precipitation and resuspension, filtration and adsorptionfor production of more pure product.

AA. method according to B above comprising addition of about 0.001 to 20 mM of a compaction agent selected from the group consisting of: basic polypeptides, polyamines, trivalent and tetravalent metal ions, or manganese chloride.

BB. The method of B above wherein the source of the lysate comprises gram-positive bacteria, yeast, eukaryotes, synthesized nucliec acids, Archaea, bacteria, protozoa, phages, other viruses, human cells, body fluids, mixtures of cells, tissues, or environmental samples.

DD. A method of performing a bioassay or separation comprising compaction precipitation, wherein a tagged probe (e.g. a fluoresceinated probe) is added to a solution containing its target, a double stranded nucleic acid is formed and this new structured hybrid nucleic acid is then selectively precipitated while the unhybridized single stranded probe is substantially left in solution.

EE. A method according to DD comprising precipitating a substantial fraction of the DNA away from contaminating RNA and protein by addition of the compaction equivalent of one volume of from 1 to 10 mM spermidine in the form of a compaction agent.

FF. A method of separating a nucleic acid-binding protein comprising compaction precipitation, wherein a lysate containing the nucleic acid-binding protein and its nucleic acid binding partner is treated with compaction agent.

The protein is substantially precipitated along with its nucleic acid binding partner, and can optionally be further purified from the precipitate.

GG. A composition of C above comprising less than about 0.1 Units endotoxin per microgram plasmid DNA (EU/ug or IE/ug). 5b.

HH. A method according to B above producing a product comprising less than 0.3 Units endotoxin per microgram plasmid DNA (EU/ug or IE/ug).

II. A composition of C above comprising less than 0.3 Units endotoxin per microgram plasmid DNA (EU/ug or IE/ug).

JJ. A composition of C above comprising less than 0.1 Units endotoxin per microgram plasmid DNA (EU/ug or IE/ug).

KK. A biotech kit comprising compaction agent and other reagents and apparatus designed for the purification of nucleic acids from lysates or synthetic solutions.

LL. A purification kit for plasmid DNA according to KK above comprised of lysis solutions, a resuspension solution, a compaction agent-based precipitation solution, a stripping solution and optionally a final resuspension solution. [based of Example 8.]

MM. A purification kit for total RNA according to KK above comprised of a lysis solution; a 1^{st} compaction precipitation solution (which may be optionally combine with the lysis solution); a 2^{nd} compaction precipitation solution; a stripping solution; and optionally a final resuspension solution. [based on Example 26.]

NN. A purification kit for chromosomal or genomic DNA according to KK above comprised of a lysis solution or solutions; a resuspension solution, a compaction agent-based precipitation solution, a stripping solution, and optionally a final resuspension solution. [based on Example 27.]

OO. A purification kit for large RNA fragments according to KK above comprised of a lysis solution; a 1^{st} compaction precipitation solution (which may be optionally combine with the lysis solution); a 2^{nd} compaction precipitation solution; a stripping solution; and optionally a final resuspension solution. [based on Example 26.]

PP. A purification kit for low molecular weight RNA fragments according to KK above comprised of a lysis solution; a 1^{st} compaction precipitation solution (which may be optionally combine with the lysis solution); a 2^{nd} compaction precipitation solution; a 3^{rd} compaction precipitation solution; a stripping solution; and optionally a final resuspension solution. [based on Example 26.]

QQ. A large-scale plasmid DNA purification kit according to KK above comprised of lysis solutions, a resuspension solution, a compaction agent-based precipitation solution, a stripping solution and optionally a final resuspension solution. [based on Example 1.]

RR. A large-scale filtration-based plasmid DNA purification kit according to QQ above comprised of lysis solutions, a resuspension solution, a compaction agent-based precipitation solution, a stripping solution and optionally a final resuspension solution. [Based on Example 23.]

SS. The use of filtration devices to enhance the speed and usability of kits listed in KK-SS above.

Reference to documents made in the specification is intended to result in such patterns or literature being expressly incorporated herein by reference.

TABLE A

| Parameter | Units | Preferred | Most Pref. |
|---|---|---|---|
| Cell Mass | | Archaea eukaryotes | Gram-neg |
| | | bacterial, Gram-negative | |
| | | Gram-positive | |
| | | phage, yeast | |
| Product | | DNA, RNA Assay | plasmid |
| | | NA-binding protein | DNA |
| | | enzymes, cosmids, | |
| | | YACs, | |
| | | Plasmid | |

Compaction Agent: basic polypeptides (e.g. polylysine),
polyamines (e.g. protamine, spermidine, spermine, putrescine, cadaverine,
etc.), trivalent and tetravalent metal ions (e.g. hexammine cobalt,
chloropentammine cobalt, chromium (III)), netropsin, distamycin, lexitropans,
DAPI (4′,6 diamino 2-phenylindol), berenil, pentamidine, manganese chloride.
Most preferred: hexammine cobalt, spermine and spermidine

| | | | |
|---|---|---|---|
| CA Conc. | mM | 0.02-20 | 0.05-10 |
| Lysing Agent: | detergent | nonionic det. | BPER for RNA; |
| (alkaline lysis | | | |
| is m.p. for | | | |
| plasmidDNA) | | | |
| Lysing Conc.: | wt % | 0.5-2 | .05-.5 |
| pH: | varies | 6-8 | 7 |
| Ionic Strength: | mM | 0-200 | 0-50 |
| (Before Compaction) | | | |
| Endotoxin Level | | >0.3 EU/mL | >0.1 EU/mL |

What is claimed is:

1. A method of preparing substantially purified DNA, without the use of nucleases or proteases, by adding an effective amount of a compaction agent to a lysate to precipitate, from said lysate, DNA having a content of RNA of less than 3% by weight.

2. A method for the production of purified DNA having a content of RNA of less than about 3% by weight, comprising in combination the following steps:
A. lysing a cell mass to liberate the nucleic acids
B. optionally precipitating some additional moieties.
C. optionally adjusting the ionic strength and/or plasmid concentration and;
D. precipitating a substantial fraction of the DNA away from contaminating RNA and protein by addition of an effective amount of a compaction agent.

3. A composition of matter comprising DNA, substantially free of added nucleases, and containing less than about 3% by weight RNA

4. A method of treatment of a mixture comprising desired RNA product and contaminating DNA comprising mechanical lysis of the mixture in the presence of a compaction agent to precipitate at least a portion of the contaminating DNA.

5. A composition of claim 3 comprising a plasmid DNA encoding proteins for use as a vaccine.

6. A method for making a biochemical assay comprising hybridizing a labeled probe to a target and thereafter precipitating the probe and the target, leaving the unhybridized probe substantially in solution.

7. A method according to claim 2 for producing ribosomal RNA, chromosomal DNA, plasmid DNA, aptamers, artificial RNA, or mRNA or other natural or synthetic nucleic acids.

8. The method of claim 1 in which the addition of the compaction agent comprises the addition of two or more different mixed compaction agents whereby improved separation efficiency results.

9. A method according to claim 1 additonally comprising stripping the compaction agent by a stripping method selected from the group comprising high salt addition and/or a pH shift.

10. A composition for the recovery of DNA comprising a mixture of combined reagents, one of which lyses and one of which precipitates DNA to clarify a cell mass.

11. A method according to claim 2 additionallly comprising a technique selected from the group consisting of: use of French cell press, addition of nonionic detergent, lysozyme addition, microfluidizer, freeze-thaw or any other relatively low ionic strength lysis technique to produce nucleic acid free lysates for later protein recovery.

12. A method according to claim 1 comprising simultaneous application of the method in parallel mini-prep procedures for a plurality of cell masses.

13. A method of assay comprising precipitating a labeled probe while it is hybridized to a target.

14. A method according to claim 2 additionally comprising a further separation step comprising one or more techniques selected from the group consisting of precipitation and resuspension, filtration and adsorptionfor production of more pure product.

15. A method according to claim 2 comprising addition of 0.001 to 20 mM of a compaction agent selected from the group consisting of: basic polypeptides, polymines, trivalent and tetravalent metal ions, or manganese chloride.

16. A method of claim 2 wherein the source of the lysate comprises gram-positive bacteria, protozoa, phages, other viruses, human cells, body fluids, mixtures of cells, tissues, or environmental samples.

17. A method of performing a bioassay or separation comprising compaction precipitation, wherein a tagged probe (e.g. a fluoresceinated probe) is added to a solution containing its target, a double stranded nucleic acid is formed and this new structured hybrid nucleic acid is then selectively precipitated while the nhybridized single stranded probe is substantially left in solution.

18. A composition of claim 3 comprising less than 0.1 Units endotoxin per microgram plasmid DNA (EU/ug or IE/ug).

19. A biotech kit comprising compaction agent and other reagents and apparatus disigned for the purification of nucleic acids from lysates or synthetic solutions.

20. Each invention described herein.
